(19) United States
(12) Patent Application Publication
(10) Pub. No.: US 2016/0362692 A1
(43) Pub. Date: Dec. 15, 2016

(54) TREATMENT OF RETINITIS PIGMENTOSA

(71) Applicant: ISIS INNOVATION LIMITED, Oxford (GB)

(72) Inventors: Robert E. MacLaren, Oxford (GB); Daniel Lipinski, Bedford (GB)

(21) Appl. No.: 14/739,183

(22) Filed: Jun. 15, 2015

(51) Int. Cl. C12N 15/113

(52) U.S. Cl. CPC C12N 15/1131; C12N 2750/14341

(57) ABSTRACT

A method of treating or preventing retinitis pigmentosa, wherein the method comprises administering an adeno-associated virus (AAV) vector comprising a nucleotide sequence encoding ciliary neurotrophic factor (CNTF) to a subject in need thereof.

### FIELD OF THE INVENTION

The present invention relates to compounds for use in the gene therapy of eye diseases. More specifically, the present invention relates to adeno-associated virus (AAV) vectors for use in the treatment of retinitis pigmentosa (RP), wherein the AAV vectors enable delivery of ciliary neurotrophic factor (CNTF) to the eye.

### BACKGROUND TO THE INVENTION

Retinitis pigmentosa (RP) is a phenotypically linked group of inherited retinal dystrophies that leads to gradual reduction in vision. RP affects approximately 1 in 3000-4000 people.

Early symptoms of RP include deterioration of night and peripheral vision. As the disease progresses, detailed, central and colour vision may also be affected. The age of onset of RP symptoms is variable, but typically between 10 and 30, and the rate of deterioration varies between individuals.

RP is commonly caused by the progressive degeneration of rod photoreceptor cells. However, the retinal pigment epithelium (RPE) and cone photoreceptor cells may also degenerate during progression of the disease.

Several compounds have been demonstrated to have some efficacy in the preservation of retinal neurons, including brain-derived neurotrophic factor (BDNF; Okoye, G. et al. (2003) J. Neurosci. 23: 4164-4172), glial cell line-derived neurotrophic factor (GDNF; McGee Sanftner, L. H. et al. (2001) Mol. Ther. 4: 622-629; Buch, P. K. et al. (2006) Mol. Ther. 14: 700-709) and ciliary neurotrophic factor (CNTF). CNTF has been shown to be protective against retinal ganglion cell death in models of oxidative stress and experimental glaucoma (Ji, J. Z. et al. (2004) Eur. J. Neurosci. 19: 265-272; Leaver, S. G. et al. (2006) Eur. J. Neurosci. 24: 3323-3332; MacLaren, R. E. et al. (2006) Exp. Eye Res. 83: 1118-1127; Maier, K. et al. (2004) Brain Pathol. 14: 378-387; Pease, M. E. et al. (2009) Invest. Ophthalmol. Vis. Sci. 50: 2194-2200).

A number of studies have demonstrated that sustained expression of CNTF following AAV-mediated gene delivery may result in preservation of photoreceptor cell bodies for a period of months in rodent models of RP (Bok, D. et al. (2002) Exp. Eye Res. 74: 719-735; Schlichtenbrede, F. C. et al. (2003) Gene Ther. 10: 523-527; Liang, F. Q. et al. (2001) Mol. Ther. 3: 241-248). However, due to the slow-progressing nature of neurodegenerative disorders such as RP, and the short term nature of these studies, it was previously unclear whether any photoreceptor protection observed would be maintained throughout the lifetime of an animal, and critically whether the preserved neurons retain function.

Moreover, although these studies observed that CNTF treatment led to anatomical preservation of photoreceptors, they concluded that AAV-mediated delivery of CNTF is not appropriate for clinical use in the treatment of RP. Specifically, the experiments (e.g. electroretinography, ERG) carried out in these studies show deterioration of visual function following vector administration (Schlichtenbrede, F. C. et al. (2003) Gene Ther. 10: 523-527).

An alternative previous approach to deliver CNTF to the eye used encapsulated cell technology, employing devices containing cells which produce CNTF. Studies on this approach have shown some success in photoreceptor preservation in the medium term, however the doses applied have been too low and too late to determine efficacy in protecting visual function (Birch, D. G. et al. Long-term follow-up of patients with retinitis pigmentosa (RP) receiving sustained-release CNTF through intraocular encapsulated cell technology implants. ARVO annual meeting (Seattle, Wash., 2013); Birch, D. G. et al. (2013) Am. J. Ophthalmol. 156: 283-292 e281). Furthermore, this technique requires significant surgical intervention to implant cell-containing devices in the eye of a subject, and the implants themselves may present problems with safety and immunogenicity.

There is currently no approved therapy to prevent the development of RP or to improve vision following the onset of the disease. Accordingly, there remains a significant need for treatments of RP, in particular to prevent deterioration in visual function or to enable improvement in the vision of affected individuals.

### SUMMARY OF THE INVENTION

The present inventors have shown that AAV-mediated expression of CNTF confers life-long protection against photoreceptor degeneration in a murine model. The inventors used repetitive retinal imaging to quantify the survival of intrinsically fluorescent cone photoreceptors in vivo. This novel approach was used to identify a specific dose range of CNTF that was high enough to halt photoreceptor degeneration without adverse effects on visual function.

Surprisingly, the present inventors have also demonstrated that surviving cone cells retain function and signal correctly to the brain. In particular, the present inventors have employed imaging of the visual cortex and assessment of visually evoked behavioural responses to discover an unexpected beneficial effect on visual function of AAV-mediated delivery of CNTF to the eye.

The present inventors utilised variable dosing in order to preserve cone photoreceptors at a stage when rod degeneration was already established, closely mimicking the ocular phenotype of many RP patients when presenting to the clinic. By combining standard assessments of electrophysiological function with visually guided behavioural testing, the inventors determined the extent of functional vision remaining following long-term CNTF treatment whilst correlating their observations to evidence of higher signal processing in the visual cortex.

In summary, the present inventors have unexpectedly found that AAV-mediated delivery of CNTF to the eye is clinically applicable to the maintenance of visual function and treatment of RP.

Accordingly, in one aspect, the present invention provides a method of treating or preventing retinitis pigmentosa, wherein the method comprises administering an adeno-associated virus (AAV) vector comprising a nucleotide sequence encoding ciliary neurotrophic factor (CNTF) to a subject in need thereof.

The AAV vector may be of any serotype (e.g. comprise any AAV serotype genome and/or capsid protein), provided that the vector is capable of infecting or transducing cells of the eye.

In one embodiment, the AAV vector comprises an AAV serotype 1, 2, 3, 4, 5, 6, 7, 8, 9, 10, or 11 genome. In another embodiment, the AAV vector comprises an AAV serotype 2, 4, 5 or 8 genome. Preferably, the AAV vector comprises an AAV serotype 2 genome.

Preferably, the AAV vector is in the form of an AAV vector particle.

In one embodiment, the AAV vector particle comprises an AAV2 genome and AAV2 capsid proteins (AAV2/2); an AAV2 genome and AAV5 capsid proteins (AAV2/5); or an AAV2 genome and AAV8 capsid proteins (AAV2/8). Preferably, the AAV vector particle comprises an AAV2 genome and AAV2 capsid proteins (AAV2/2).

The AAV vector particle of the invention may be a chimeric, shuffled or capsid-modified derivative of one or more naturally occurring AAVs. In particular, the AAV vector particle may comprise capsid protein sequences from different serotypes, clades, clones or isolates of AAV within the same vector (i.e. a pseudotyped vector). Thus, in one embodiment the AAV vector is in the form of a pseudotyped AAV vector particle.

In one embodiment, the CNTF is human CNTF.

In another embodiment, the CNTF-encoding nucleotide sequence is selected from the group consisting of:

(a) a nucleotide sequence having at least 70% identity to SEQ ID NO: 1; and

(b) a nucleotide sequence encoding an amino acid sequence having at least 70% identity to SEQ ID NO: 2,

wherein the protein encoded by the nucleotide sequence substantially retains the natural function of the protein represented by SEQ ID NO: 2.

In another embodiment, the CNTF-encoding nucleotide sequence comprises a nucleotide sequence that has at least 70%, 80%, 90%, 95%, 96%, 97%, 98% 99% or 100% identity to SEQ ID NO: 1, wherein the protein encoded by the nucleotide sequence substantially retains the natural function of the protein represented by SEQ ID NO: 2.

In another embodiment, the CNTF-encoding nucleotide sequence comprises a nucleotide sequence encoding an amino acid sequence that has at least 70%, 80%, 90%, 95%, 96%, 97%, 98% 99% or 100% identity to SEQ ID NO: 2, wherein the amino acid sequence substantially retains the natural function of the protein represented by SEQ ID NO: 2.

In one embodiment, the AAV vector comprises a secretion signal sequence operably linked to the CNTF-encoding nucleotide sequence. Preferably, the secretion signal sequence is a human neuronal growth factor (NGF) secretion signal sequence.

In one embodiment, the secretion signal sequence comprises a nucleotide sequence having at least 70%, 80%, 90%, 95%, 96%, 97%, 98% 99% or 100% identity to SEQ ID NO: 3 or 7, wherein the peptide encoded by the nucleotide sequence causes the CNTF to which it is operably linked to be secreted from the cell in which it is expressed.

In one embodiment, the AAV vector is administered to the eye of a subject by subretinal, direct retinal or intravitreal injection. Preferably, the AAV vector is administered to the eye of a subject by subretinal injection. The subretinal injection may be performed using the two-step subretinal injection method described herein.

In one embodiment, the AAV vector is administered to a subject in a single dose.

The AAV vector may, for example, be in a suspension at a concentration of about 1-2×10¹¹, 1-2×10¹² or 1-2×10¹³ genome particles (gp) per mL. Thus a dose of AAV vector of about 2×10¹⁰ gp may, for example, be administered by injecting about a 10 μL dose of AAV vector at a concentration of about 2×10¹² gp per mL. The skilled person is readily able to adjust the dose, volume and concentration of the AAV vector as necessary.

The volume of the AAV vector administered may be, for example, about 1-500 μL, for example about 10-500, 50-500, 100-500, 200-500, 300-500, 400-500, 50-250, 100-250, 200-250, 50-150, 1-100 or 1-10 μL. The volume may be, for example, about 1, 2, 5, 10, 50, 100, 150, 200, 250, 300, 350, 400, 450 or 500 μL. Preferably, the volume of the AAV vector composition injected is about 100 μL.

In one embodiment, the AAV vector is administered at a dosage of at least 2×10⁹, 2×10¹⁰, 2×10¹¹ or 2×10¹² gp per eye. In another embodiment, the AAV vector is administered at a dosage of about 1-2×10⁹, 1-2×10¹⁰, 1-2×10¹¹ or 1-2×10¹² gp per eye. Preferably, the AAV vector is administered at a dosage of about 2×10¹¹ gp per eye, preferably by subretinal injection.

The present inventors have surprisingly found that treatment of retinitis pigmentosa using the AAV vector of the present invention can be effective (e.g. in maintaining visual function) even if most or all of the rod cells have degenerated, for example by protecting the surviving cone cells. Accordingly in one embodiment, the eye to be treated comprises less than about 100 million, 50 million, 20 million, 10 million, 9 million, 8 million, 7 million, 6 million, 5 million, 4 million, 3 million, 2 million, 1 million, 500000, 250000, 100000 or 50000 rod cells at the time of administration of the AAV vector. In another embodiment, the subject substantially lacks rod cells in the eye to be treated at the time of administration of the AAV vector. In another embodiment, the eye to be treated comprises zero rod cells at the time of administration of the AAV vector.

In one embodiment, photoreceptor cell degeneration due to retinitis pigmentosa is substantially prevented for the lifetime of the subject. The photoreceptor cells may comprise cone cells and/or rod cells, preferably cone and rod cells. In another embodiment, less than about 90%, 80%, 70%, 60%, 50%, 40%, 30%, 20%, 15%, 10%, 9%, 8%, 7%, 6%, 5%, 4%, 3%, 2% or 1% of the number of photoreceptor cells (e.g. cone cells and/or rod cells) present in the treated eye at the time of administration of the AAV vector subsequently degenerate due to retinitis pigmentosa over the lifetime of the subject.

In another embodiment, cone cell degeneration due to retinitis pigmentosa is substantially prevented for the lifetime of the subject. In another embodiment, less than about 90%, 80%, 70%, 60%, 50%, 40%, 30%, 20%, 15%, 10%, 9%, 8%, 7%, 6%, 5%, 4%, 3%, 2% or 1% of the number of cone cells present in the treated eye at the time of administration of the AAV vector subsequently degenerate due to retinitis pigmentosa over the lifetime of the subject. Preferably, the surviving cells remain functional.

In another aspect, the present invention provides a method of reducing photoreceptor cell death in a subject suffering from or at risk of developing retinitis pigmentosa, wherein the method comprises administering an adeno-associated virus (AAV) vector comprising a nucleotide sequence encoding ciliary neurotrophic factor (CNTF) to a subject in need thereof.

The AAV vector, CNTF and mode (e.g. method and dosage) of administration may be as described herein.

In one embodiment, visual function is substantially restored or maintained in the treated eye. Visual function (e.g. as determined by a test of visual function described herein) may, for example, be restored to about the same level in an affected eye as existed before the onset of retinitis pigmentosa. Alternatively, visual function may, for example, be maintained at about the same level in a healthy subject at risk of developing retinitis pigmentosa, or in a subject already suffering from retinitis pigmentosa (e.g. substantially no deterioriation or further deterioration of visual function occurs as a result of retinitis pigmentosa following the administration of the AAV vector of the invention).

If left untreated, most or all rod cells may degenerate (e.g. die) over time as a result of retinitis pigmentosa. Cone cells may also degenerate during progression of the disease.

In one embodiment, photoreceptor cell degeneration due to retinitis pigmentosa is substantially prevented for the lifetime of the subject. The photoreceptor cells may comprise cone cells and/or rod cells, preferably cone and rod cells. In another embodiment, less than about 90%, 80%, 70%, 60%, 50%, 40%, 30%, 20%, 15%, 10%, 9%, 8%, 7%, 6%, 5%, 4%, 3%, 2% or 1% of the number of photoreceptor cells (e.g. cone cells and/or rod cells) present in the treated eye at the time of administration of the AAV vector subsequently degenerate due to retinitis pigmentosa over the lifetime of the subject.

In another embodiment, cone cell degeneration due to retinitis pigmentosa is substantially prevented for the lifetime of the subject. In another embodiment, less than about 90%, 80%, 70%, 60%, 50%, 40%, 30%, 20%, 15%, 10%, 9%, 8%, 7%, 6%, 5%, 4%, 3%, 2% or 1% of the number of cone cells present in the treated eye at the time of administration of the AAV vector subsequently degenerate due to retinitis pigmentosa over the lifetime of the subject. Preferably, the surviving cells remain functional.

In one embodiment, the eye to be treated comprises less than about 100 million, 50 million, 20 million, 10 million, 9 million, 8 million, 7 million, 6 million, 5 million, 4 million, 3 million, 2 million, 1 million, 500000, 250000, 100000 or 50000 rod cells at the time of administration of the AAV vector. In another embodiment, the subject substantially lacks rod cells in the eye to be treated at the time of administration of the AAV vector. In another embodiment, the eye to be treated comprises zero rod cells at the time of administration of the AAV vector.

In another aspect, the present invention provides an adeno-associated virus (AAV) vector for use in the treatment or prevention of retinitis pigmentosa, wherein the AAV vector comprises a nucleotide sequence encoding ciliary neurotrophic factor (CNTF).

The AAV vector, CNTF, mode (e.g. method and dosage) and effect of administration, and the subject to be treated may be as described herein.

In another aspect, the present invention provides an adeno-associated virus (AAV) vector for use in the reduction of photoreceptor cell death in a subject suffering from or at risk of developing retinitis pigmentosa, wherein the AAV vector comprises a nucleotide sequence encoding ciliary neurotrophic factor (CNTF).

The AAV vector, CNTF, mode (e.g. method and dosage) and effect of administration, and the subject to be treated may be as described herein.

In another aspect, the present invention provides a genetically modified animal for analysing cone cell survival and/or function, wherein the animal has been genetically modified to comprise a detectable marker in its cone cells.

In one embodiment, the detectable marker is a fluorescent protein, such as green fluorescent protein.

In another embodiment, the animal is a mammal, preferably a mouse.

In another embodiment, the animal is also a disease model animal. Preferably, the animal has been genetically modified to provide a model for retinitis pigmentosa, for example the animal may be homozygous for the knockout of rhodopsin.

### DESCRIPTION OF THE DRAWINGS

FIG. 1

(a) Autofluorescence (AF) image of Rho^{-/-TgOPN1LW-EGFP+/-} mouse fundus showing cone photoreceptors represented as punctate white dots. (a′) Increase magnification AF image demonstrating a distinctive group of cone photoreceptors (aligned β to γ) and individual cones (white arrows) at the nasal vascular bifurcation (a, dotted line). (b) Near infrared (NIR) fundus image detailing the retina vasculature and (b′) Magnified NIR image detailing the nasal vascular bifurcation (a, dotted line). (c) Flat mounted post-mortem retina with retinal vasculature counterstained with lectin. (c′) Magnified image of the retinal flat mount demonstrating the distribution of cone photoreceptors (green dots) at the nasal bifurcation (a, dotted line). Note that the pattern of cones in panel (c′) correlates precisely with that observed by in vivo AF imaging (a′); including a distinctive group of cone photoreceptors (aligned (3 to γ) and individual cones (white arrows). (d-g) Repetitive AF fundus imaging of a Rho^{-/-TgOPN1LW-EGFP+/-} mouse treated with high dose rAAV2/2.hCNTF from postnatal week (PW) 8 to PW30. (d′-g′) Magnified AF images showing how retinal vascular 1 lifetime of each animal. Cone photoreceptors (white dots) can be quantified (red dot overlay) within the ROI allowing highly reproducible longitudinal assessment of cone survival. Survival is expressed as a function (%) of cone photoreceptor numbers at baseline (PW8). All scale bars ˜100 μm. (h) Survival of cone photoreceptors expressed as a function (%) of cone photoreceptor numbers at baseline (PW8) for each treatment group: high dose (dark blue), medium dose (bright blue), low dose (pale blue) or PBS sham (grey). hCNTF treatment has a significant effect on cone photoreceptor survival: *=p<0.05; **=p<0.01; ***=p<0.001, two-way repeated measures ANOVA with dose and time as factors. High dose group n=5, medium dose group n=6, low dose group n=8, at PW30.

FIG. 2

Near infrared reflectance (NIR) and autofluorescence (AF) fundus imaging of high dose rAAV2/2.hCNTF-treated and PBS sham-treated mice at PW8 and PW30. Sham-treated controls demonstrate areas of increased reflectance indicative of retinal pigment epithelium (RPE) atrophy (a, c) and loss of EGFP-expressing cone photoreceptors (b, d) by PW30. High dose rAAV2/2.hCNTF treated eyes show no signs of RPE atrophy (e, g) and minimal loss of cone photoreceptors (f, h) over the same period. Representative histology of PBS sham treated (i) retina at PW30 demonstrates an absence of both rod and cone photoreceptors. Histology of high dose rAAV2/2.hCNTF treated (j-o) retina at PW30 demonstrating preservation of cone (GFP-expressing) and rod (non GFP-expressing) photoreceptors in the outer nuclear layer (ONL). (j-k) Cone photoreceptors (l-o) express Mws-opsin (arrow head) in the correct cellular compartment (outer segment; OS) with no evidence of opsin mislocalisation in the cell body (*). Cone photoreceptor morphology indicates compression between the inner nuclear layer (INL) and RPE due to the loss of multiple nuclear rows following rod degeneration.

FIG. 3

(a-b) change in cortical blood flow (CBF) in untreated Rho^{-/-TgOPN1LW-EGFP+/-} mice at PW6 demonstrating that laser speckle imaging (LSI) detects changes in micro-vascular blood flow primarily in the visual cortex following stimulation with a 510 nm flicker stimulus. (c) Schematic representation of the visual pathway in mice demonstrating that visual input is primarily processed in the visual cortex located in the hemisphere contralateral to the eye stimulated. White arrow=superior sagittal sinus; arrow head=lambda; OS=oculus sinister (left); OD=oculus dexter (right). Changes in CBF were examined in (d-e) medium and (g-i) high dose treated Rho^{-/-TgOPN1LW-EGFP+/-} mice at PW30 following independent stimulation of

PBS-treated (d, g) or rAAV2/2.hCNTF-treated (e, h) eyes, revealing significant differences in CBF from high dose treated eyes (i) compared to sham controls. ns=not significant; *=p<0.05, Wilcoxon matched-paired signed rank test. LSI: high dose n=3; medium dose n=6.

FIG. 4

(a) Schematic representation of the optomotor response (OMR) in relation to the direction of the drum's rotation. (b) Number of head-tracking responses (three tests per animal) from individual mice at PW30. Error=SEM; X=zero responses; L=low dose, M=medium dose, H=high dose (c) Mean group responses to OMR testing at PW30 demonstrating significantly greater number of headtracks from medium (bright blue) and high dose (dark blue) compared to paired PBS sham-treated controls. ns=not significant, ***=p<0.001, one way ANOVA. Low dose n=5, medium dose n=6, high dose n=4.

FIG. 5

(a) Hierarchical clustering of transcriptome output from low, medium and high dose rAAV2/2.hCNTF treated mice showing altered gene expression relative to paired PBS sham-treated controls (n=4 mice per group) at PW30, where the solid blue line in each column represents fold change compared to baseline (dotted blue line). Two clusters relating to genes with the highest transcriptional changes are enlarged: the upper panel shows down-regulation of genes involved in ion transport and visual cycle; the lower panel shows widespread up-regulation of genes encoding serine-, cysteine- and metallopeptidase inhibitors. (b) Clustering of genes based function using Genome Search Meta Analysis (GMSA) ontology visualised using the enrichment map plugin for cytoscape.

FIG. 6

Detection of hCNTF protein levels by ELISA from (a) media aspirated from HEK293 cells following transduction with rAAV2/2.hCNTF, or (b) from rAAV2/2.hCNTF-injected eyes, showing robust secretion of hCNTF and the ability to roughly control dose by altering the number of vector particles administered. (c-j) Repetitive AF fundus imaging of a Rho^{-/-TgOPN1LW-EGFP+/-} mouse treated with high dose rAAV2/2.hCNTF from PW8 to PW30. (c′-j′) Magnified AF images showing how retinal vascular landmarks can be used to define a region of interest (ROI) that remains constant throughout the animal's lifetime. Cone photoreceptors (white dots) can be quantified (red dot overlay) within the ROI allowing highly reproducible longitudinal assessment of cone survival. Survival is expressed as a function (%) of cone photoreceptor numbers at baseline (PW8). All scale bars ˜100 μm. *=p<0.05, Mann-Whitney U test; n=3 wells per group (a); n=3 eyes per group (b).

FIG. 7

Representative near infrared (NIR) and autofluorescence (AF) fundus images of Rho^{-/-TgOPN1LW-EGFP+/-} mice assigned to each of the treatment groups: (a) high dose; (b) medium dose; or (c) low dose rAAV2/2.hCNTF; or (d) PBS sham. Doses are given as genome particles (gp) administered per eye in a 2 μL volume. Cone photoreceptors are observed on the autofluorescence (AF) mode as punctuate white dots. RPE atrophy can be observed as areas of increased brightness by NIR imaging, particularly at later time points in low dose (c vii-viii) and PBS sham (d vi-viii) treatment groups.

FIG. 8

Representative electroretinography (ERG) traces of Rho^{-/-TgOPN1LW-EGFP+/-} mice treated in one eye with (a) low (pale blue), (c) medium (bright blue) or (e) high dose rAAV2/2.hCNTF (dark blue) compared to the paired PBS sham eyes (grey). (b, d, f) Measurement of the photopic b-wave at PW8, 10 and 12 in response to a 1 Hz 25 cd·s/m² flash stimuli (30 cd/m² polychromatic white light background) showing b-wave amplitude. The photopic b-wave amplitude was absent or below recordable levels in all groups by week 12. *=p<0.05; **=p<0.01; ***=p<0.001, multiple t-tests with Bonferroni post-hoc correction. High dose group n=6, medium dose group n=6, low dose group n=8, at PW12.

FIG. 9

(a) Correlation of cone photoreceptor survival with OMR score for individual medium and high dose-treated mice at PW30; solid line=linear regression, dotted lines=95% confidence intervals. r²=0.80, F=33, p=0.0004. (b) Graphical representation demonstrating transcriptome analysis in high dose treated eyes versus PBS sham controls; 23365 transcripts (grey dots) were assessed for differential expression based on log 2-fold change versus mean normalised counts. 1533 genes (red dots) were observed to have significantly different expression levels in high dose rAAV2/2.hCNTF treated eyes compared to paired sham-treated controls.

### TABLE 1

Selected genes grouped by gene ontology. The gene symbol (UCSC, mm9), description, fold-change, log 2 fold change and significance is given for each gene. Upregulated genes=blue shading; downregulated=red shading.

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

In one aspect, the present invention provides a method of treating or preventing retinitis pigmentosa, wherein the method comprises administering an adeno-associated virus (AAV) vector comprising a nucleotide sequence encoding ciliary neurotrophic factor (CNTF) to a subject in need thereof.

### Retinitis Pigmentosa (RP)

Retinitis pigmentosa (RP) is a phenotypically linked group of inherited retinal dystrophies which is commonly caused by the progressive degeneration of rod photoreceptor cells. The retinal pigment epithelium (RPE) and cone photoreceptor cells may also degenerate during progression of the disease.

RP is characterised in clinical appearance by changes in the pigment of the retina, which may be accompanied by arteriolar attentuation and optic nerve atrophy. Changes in the retina may result from dispersion and aggregation of the retinal pigment. This may give rise to an appearance ranging from granular or mottled to distinctive focal aggregates resembling bone spicules. Black or dark brown star-shaped concentrations of pigment may appear. Furthermore, pigmentation limited to one quadrant of the retina, abnormalities which appear to be radiating out from the disc and changes associated with severe vasculopathy may be observed.

The treatment or prevention of RP described herein may reduce or prevent the appearance of the RP phenotype described above. It may result in protection of the photoreceptor cells, such as the cone cells, from degeneration. Preferably, the treatment protects both cone and rod cells from degeneration.

Numbers of rods and cones can be estimated by the skilled person in the clinic using techniques such as adaptive optics, autofluorescence and optical coherence tomography (OCT) scans.

Preferably, the treatment of RP enables maintenance or improvement in visual function.

Visualisation of the appearance of a retina and assessment of visual function may be readily carried out by the skilled person. For example, visual function tests that might be carried out by the skilled person include best corrected visual acuity, visual field testing, microperimetry, colour vision, dark adaptometry, electroretinography and cone flicker fusion tests. As used herein, “maintenance or improvement in visual function” is to be understood as the maintenance of substantially the same level or an improvement in the level of vision as assessed by one or more such test of visual function, when the vision in a treated eye is compared before and after the methods of the invention have been performed.

### Structure of the Eye

The medicaments disclosed herein may be delivered to a mammalian, preferably human eye in relation to the treatment or prevention of retinitis pigmentosa (RP).

The person skilled in the treatment of diseases of the eye will have a detailed and thorough understanding of the structure of the eye. However, the following structures of particular relevance to the present invention are described.

### Retina

The retina is the multi-layered membrane which lines the inner posterior chamber of the eye and senses an image of the visual world which is communicated to the brain via the optic nerve. In order from the inside to the outside of the eye, the retina comprises the layers of the neurosensory retina and retinal pigment epithelium, with the choroid lying outside the retinal pigment epithelium.

### Neurosensory Retina and Photoreceptor Cells

The neurosensory retina harbours the photoreceptor cells that directly sense light. It comprises the following layers: internal limiting membrane (ILM); nerve fibre layer; ganglion cell layer; inner plexiform layer; inner nuclear layer; outer plexiform layer; outer nuclear layer (nuclei of the photoreceptors); external limiting membrane (ELM); and photoreceptors (inner and outer segments) of the rods and cones.

The skilled person will have a detailed understanding of photoreceptor cells. Briefly, photoreceptor cells are specialised neurons located in the retina that convert light into biological signals. Photoreceptor cells comprise rod and cone cells, which are distributed differently across the retina.

Rod cells are distributed mainly across the outer parts of the retina. They are highly sensitive and provide for vision at low light levels. There are on average about 125 million rod cells in a normal human retina.

Cone cells are found across the retina, but are particular highly concentrated in the fovea, a pit in the neurosensory retina that is responsible for central high resolution vision. Cone cells are less sensitive than rod cells. There are on average about 6-7 million cone cells in a normal human retina.

### Retinal Pigment Epithelium

The retinal pigment epithelium (RPE) is a pigmented layer of cells located immediately to the outside of the neurosensory retina. The RPE performs a number of functions, including transport of nutrients and other substances to the photoreceptor cells, and absorption of scattered light to improve vision.

### Choroid

The choroid is the vascular layer situated between the RPE and the outer sclera of the eye. The vasculature of the choroid enables provision of oxygen and nutrients to the retina.

### Ciliary Neurotrophic Factor (CNTF)

Ciliary neurotrophic factor (CNTF) is a polypeptide hormone and nerve growth factor which has been shown to be a survival factor for neurons and oligodendrocytes. In addition, CNTF has been found to promote neurotransmitter synthesis and neurite outgrowth in certain neural populations.

In one embodiment of the present invention, the CNTF is human CNTF.

In one embodiment, the nucleotide sequence encoding CNTF is the sequence deposited under NCBI Accession No. NM_000614.

In another embodiment, the nucleotide sequence encoding CNTF is:

(SEQ ID NO: 1)
ATGGCTTTCACAGAGCATTCACCGCTGACCCCTCACCGTCGGGACCTCTG
TAGCCGCTCTATCTGGCTAG
CAAGGAAGATTCGTTCAGACCTGACTGCTCTTACGGAATCCTATGTGAAG
CATCAGGGCCTGAACAAGAA
CATCAACCTGGACTCTGCGGATGGGATGCCAGTGGCAAGCACTGATCAGT
GGAGTGAGCTGACCGAGGCA
GAGCGACTCCAAGAGAACCTTCAAGCTTATCGTACCTTCCATGTTTTGTT
GGCCAGGCTCTTAGAAGACC
AGCAGGTGCATTTTACCCCAACCGAAGGTGACTTCCATCAAGCTATACAT
ACCCTTCTTCTCCAAGTCGC
TGCCTTTGCATACCAGATAGAGGAGTTAATGATACTCCTGGAATACAAGA
TCCCCCGCAATGAGGCTGAT
GGGATGCCTATTAATGTTGGAGATGGTGGTCTCTTTGAGAAGAAGCTGTG
GGGCCTAAAGGTGCTGCAGG
AGCTTTCACAGTGGACAGTAAGGTCCATCCATGACCTTCGTTTCATTTCT
TCTCATCAGACTGGGATCCC
AGCACGTGGGAGCCATTATATTGCTAACAACAAGAAAATGTAG

In one embodiment, the amino acid sequence of CNTF is the sequence deposited under NCBI Accession No. NP_000605.

In another embodiment, the amino acid sequence of CNTF is:

(SEQ ID NO: 2)
MAFTEHSPLTPHRRDLCSRSIWLARKIRSDLTALTESYVKHQGLNKNINL
DSADGMPVASTDQWSELTEA
ERLQENLQAYRTFHVLLARLLEDQQVHFTPTEGDFHQAIHTLLLQVAAFA
YQIEELMILLEYKIPRNEAD
GMPINVGDGGLFEKKLWGLKVLQELSQWTVRSIHDLRFISSHQTGIPARG
SHYIANNKKM

The nucleotide sequence encoding CNTF of the present invention may, for example, comprise a nucleotide sequence that has at least 70%, 80%, 90%, 95%, 96%, 97%, 98% 99% or 100% identity to SEQ ID NO: 1, wherein the protein encoded by the nucleotide sequence substantially retains the natural function of the protein represented by SEQ ID NO: 2.

The nucleotide sequence encoding CNTF of the present invention may, for example, encode an amino acid sequence that has at least 70%, 80%, 90%, 95%, 96%, 97%, 98% 99% or 100% identity to SEQ ID NO: 2, wherein the amino acid sequence substantially retains the natural function of the protein represented by SEQ ID NO: 2.

Preferably, the nucleotide sequence of the present invention encodes a protein which assists in providing similar or higher prevention of:

(a) the clinical appearance of the retinal pigment changes that are associated with RP;

(b) photoreceptor (e.g. cone cell, preferably cone and rod cell) cell death; and/or

(c) deterioration in visual function

in a subject suffering from or at risk of developing RP compared to the protein of SEQ ID NO: 2.

### Secretion Signal Sequence

In one embodiment of the present invention, the AAV vector comprises a secretion signal sequence operably linked to the CNTF-encoding nucleotide sequence.

Signal sequences are generally short peptides at the N-terminus of a protein that target the protein to the secretory pathways of a cell. As used herein, a “secretion signal sequence” is a peptide that promotes the secretion of a protein (e.g. CNTF) from a cell, or the nucleotide sequence encoding that peptide, as appropriate.

The peptide of the secretion signal sequence may be cleaved from the protein upon its secretion from the cell, resulting in a mature form of that protein.

In one embodiment, the secretion signal sequence is a human neuronal growth factor (NGF) secretion signal sequence.

In another embodiment, the secretion signal sequence is the signal sequence of the NGF nucleotide sequence deposited under NCBI Accession No. NM_002506 (e.g. the sequence corresponding to nucleotides 170-223 of the mRNA deposited under that accession number).

In another embodiment, the secretion signal sequence is:

(SEQ ID NO: 3)
ATGTCCATGTTGTTCTACACTCTGATCACAGCTTTTCTGATCGGCATACA
GGCG

In another embodiment, the secretion signal sequence is:

(SEQ ID NO: 7)
ATGTCCATGTTGTTCTACACTCTGATCACAGCTTTTCTGATCGGCATACA
GGGGAACCACACTCA

The secretion signal sequence of the present invention may, for example, comprise a nucleotide sequence having at least 70%, 80%, 90%, 95%, 96%, 97%, 98% 99% or 100% identity to SEQ ID NO: 3 or 7, wherein the peptide encoded by the nucleotide sequence causes the CNTF to which it is operably linked to be secreted from the cell in which it is expressed.

By “operably linked”, it is to be understood that the individual components are linked together in a manner which enables them to carry out their function substantially unhindered (e.g. a secretion signal sequence may be operably linked to a nucleotide of interest to promote secretion of the protein encoded by the nucleotide of interest from a cell; or a promoter may be operably linked to a nucleotide of interest to promote expression of the nucleotide of interest in a cell).

SEQ ID NOs: 8 and 9 correspond to example nucleotide and amino acid sequences, respectively, of a human NGF secretion signal sequence operably linked to a human CNTF sequence.

(SEQ ID NO: 8)
ATGTCCATGTTGTTCTACACTCTGATCACAGCTTTTCTGATCGGCATACA
GGCGGAACCACACTCAGCTT
TCACAGAGCATTCACCGCTGACCCCTCACCGTCGGGACCTCTGTAGCCGC
TCTATCTGGCTAGCAAGGAA
GATTCGTTCAGACCTGACTGCTCTTACGGAATCCTATGTGAAGCATCAGG
GCCTGAACAAGAACATCAAC
CTGGACTCTGCGGATGGGATGCCAGTGGCAAGCACTGATCAGTGGAGTGA
GCTGACCGAGGCAGAGCGAC
TCCAAGAGAACCTTCAAGCTTATCGTACCTTCCATGTTTTGTTGGCCAGG
CTCTTAGAAGACCAGCAGGT
GCATTTTACCCCAACCGAAGGTGACTTCCATCAAGCTATACATACCCTTC
TTCTCCAAGTCGCTGCCTTT
GCATACCAGATAGAGGAGTTAATGATACTCCTGGAATACAAGATCCCCCG
CAATGAGGCTGATGGGATGC
CTATTAATGTTGGAGATGGTGGTCTCTTTGAGAAGAAGCTGTGGGGCCTA
AAGGTGCTGCAGGAGCTTTC
ACAGTGGACAGTAAGGTCCATCCATGACCTTCGTTTCATTTCTTCTCATC
AGACTGGGATCCCAGCACGT
GGGAGCCATTATATTGCTAACAACAAGAAAATGTAG
the human NGF secretion signal sequence is
underlined
(SEQ ID NO: 9)
MSMLFYTLITAFIGIQAEPHSAFTEHSPLTPHRRDLCSRSIWLARKIRSDLT
ALTESYVKHQGLNKNIN
LDSADGMPVASTDQWSELTEAERLQENLQAYRTFHVLLARLLEDQQVHFTPT
EGDFHQAIHTLLLQVAAF
AYQIEELMILLEYKIPRNEADGMPINVGDGGLFEKKLWGLKVLQELSQWTVR
SIHDLRFISSHQTGIPAR
GSHYIANNKKM
the human NGF secretion signal sequence is
underlined

In one embodiment of the present invention, the AAV vector comprises a nucleotide sequence having at least 70%, 80%, 90%, 95%, 96%, 97%, 98% 99% or 100% identity to SEQ ID NO: 8, wherein the protein encoded by the nucleotide sequence substantially retains the natural function of the protein represented by SEQ ID NO: 9.

In another embodiment of the present invention, the AAV vector comprises a nucleotide sequence encoding an amino acid sequence that has at least 70%, 80%, 90%, 95%, 96%, 97%, 98% 99% or 100% identity to SEQ ID NO: 9, wherein the amino acid sequence substantially retains the natural function of the protein represented by SEQ ID NO: 9.

### Vectors

A vector is a tool that allows or facilitates the transfer of an entity from one environment to another.

The vectors used in the present invention may include a promoter for the expression of a polynucleotide and optionally a regulator of the promoter.

### Adeno-Associated Virus (AAV) Vectors

The vector of the present invention is an adeno-associated virus (AAV) vector. Preferably, the AAV vector is in the form of an AAV vector particle.

Methods of preparing and modifying viral vectors and viral vector particles, such as those derived from AAV, are well known in the art and can be readily adapted by the skilled person to the required purpose.

The AAV vector may comprise an AAV genome or a derivative thereof.

An AAV genome is a polynucleotide sequence which encodes functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype, isolate or Glade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the present invention. Thus, in one embodiment of the present invention, the AAV vector particle is an AAV1, AAV2, AAV3, AAV4, AAV5, AAV6, AAV7, AAV8, AAV9, AAV10, AAV11, Rec2 or Rec3 AAV vector particle.

A preferred serotype of AAV for use in the invention is AAV2.

Other serotypes of particular interest for use in the invention include AAV4, AAV5 and AAV8 which efficiently transduce tissue in the eye, such as the retinal pigment epithelium.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, Glade, clone or isolate of AAV for use in the present invention on the basis of their common general knowledge. For instance, the AAV5 capsid has been shown to transduce primate cone photoreceptors efficiently as evidenced by the successful correction of an inherited colour vision defect (Mancuso et al. (2009) Nature 461: 784-7).

The AAV serotype determines the tissue specificity of infection (or tropism) of an AAV virus. Accordingly, preferred AAV serotypes for use in AAVs administered to patients in accordance with the invention are those which have natural tropism for or a high efficiency of infection of target cells within the eye. In one embodiment, AAV serotypes for use in the present invention are those which infect cells of the neurosensory retina, retinal pigment epithelium and/or choroid.

Typically, the AAV genome of a naturally derived serotype, isolate or Glade of AAV comprises at least one inverted terminal repeat sequence (ITR). An ITR sequence acts in cis to provide a functional origin of replication and allows for integration and excision of the vector from the genome of a cell. In preferred embodiments, one or more ITR sequences flank the nucleotide sequence encoding the CNTF. The AAV genome typically also comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle. Capsid variants are discussed below.

A promoter will be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters are generally used to express the rep gene, while the p40 promoter is generally used to express the cap gene.

As discussed above, the AAV genome used in the vector of the invention may therefore be the full genome of a naturally occurring AAV. For example, a vector comprising a full AAV genome may be used to prepare an AAV vector or vector particle in vitro. However, while such a vector may in principle be administered to patients, this will rarely be done in practice. Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The one or more ITRs will preferably flank the nucleotide sequence encoding the CNTF at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector).

Chimeric, shuffled or cap sid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population. An example might include the use of RGD peptide to block uptake in the retinal pigment epithelium and thereby enhance transduction of surrounding retinal tissues (Cronin et al. (2008) ARVO Abstract: D1048). The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al., referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The vector of the invention may take the form of a nucleotide sequence comprising an AAV genome or derivative thereof and a sequence encoding the CNTF transgene or a variant thereof.

The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. The AAV particle also includes chemically modified forms bearing ligands adsorbed to the capsid surface. For example, such ligands may include antibodies for targeting a particular cell surface receptor.

Thus, for example, the AAV particles of the invention include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8).

### Promoters and Regulatory Sequences

The vector of the invention may also include elements allowing for the expression of the CNTF transgene in vitro or in vivo. These may be referred to as expression control sequences. Thus, the vector typically comprises expression control sequences (e.g. comprising a promoter sequence) operably linked to the nucleotide sequence encoding the transgene.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, it is preferred that the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium.

Preferred promoters include the chicken beta-actin (CBA) promoter, optionally in combination with a cytomegalovirus (CMV) enhancer element. An example promoter for use in the invention is a hybrid CBA/CAG promoter, for example the promoter used in the rAVE expression cassette (GeneDetect.com). A further example promoter for use in the invention has the sequence:

(SEQ ID NO: 4)
ATTGACGTCAATAATGACGTATGTTCCCATAGTAACGCCAATAGGGACTT
TCCATTGACGTCAATGGGTG
GAGTATTTACGGTAAACTGCCCACTTGGCAGTACATCAAGTGTATCATAT
GCCAAGTACGCCCCCTATTG
ACGTCAATGACGGTAAATGGCCCGCCTGGCATTATGCCCAGTACATGACC
TTATGGGACTTTCCTACTTG
GCAGTACATCTACGTATTAGTCATCGCTATTACCATGGTCGAGGTGAGCC
CCACGTTCTGCTTCACTCTC
CCCATCTCCCCCCCCTCCCCACCCCCAATTTTGTATTTATTTATTTTTTA
ATTATTTTGTGCAGCGATGG
GGGCGGGGGGGGGGGGGGGGCGCGCGCCAGGCGGGGCGGGGCGGGGCGAG
GGGCGGGGCGGGGCGAGGCG
GAGAGGTGCGGCGGCAGCCAATCAGAGCGGCGCGCTCCGAAAGTTTCCTT
TTATGGCGAGGCGGCGGCGG
CGGCGGCCCTATAAAAAGCGAAGCGCGCGGCGGGCGGGAGTCGCTGCGCG
CTGCCTTCGCCCCGTGCCCC
GCTCCGCCGCCGCCTCGCGCCGCCCGCCCCGGCTCTGACTGACCGCGTTA
CTCCCACAGGTGAGCGGGCG
GGACGGCCCTTCTCCTCCGGGCTGTAATTAGCGCTTGGTTTAATGACGGC
TTGTTTCTTTTCTGTGGCTG
CGTGAAAGCCTTGAGGGGCTCCGGGAGGGCCCTTTGTGCGGGGGGAGCGG
CTCGGGGCTGTCCGCGGGGG
GACGGCTGCCTTCGGGGGGGACGGGGCAGGGCGGGGTTCGGCTTCTGGCG
TGTGACCGGCGGCTCTAGAG
CCTCTGCTAACCATGTTCATGCCTTCTTCTTTTTCCTACAGCTCCTGGGC
AACGTGCTGGTTATTGTGCT
GTCTCATCATTTTGGCAAAGAATT

Examples of promoters based on human sequences that would induce retina-specific gene expression include rhodospin kinase for rods and cones (Allocca et al. (2007) J. Virol. 81: 11372-80), PR2.1 for cones only (Mancuso et al. (2009) Nature 461: 784-7) and/or RPE65 for the retinal pigment epithelium (Bainbridge et al. (2008) N. Engl. J. Med. 358: 2231-9).

The vector of the invention may also comprise one or more additional regulatory sequences which may act pre- or post-transcriptionally. The regulatory sequence may be part of the native transgene locus or may be a heterologous regulatory sequence. The vector of the invention may comprise portions of the 5′-UTR or 3′-UTR from the native transgene transcript.

Regulatory sequences are any sequences which facilitate expression of the transgene, i.e. act to increase expression of a transcript, improve nuclear export of mRNA or enhance its stability. Such regulatory sequences include for example enhancer elements, postregulatory elements and polyadenylation sites. A preferred polyadenylation site is the Bovine Growth Hormone poly-A signal which may be as shown below:

(SEQ ID NO: 5)
TCGCTGATCAGCCTCGACTGTGCCTTCTAGTTGCCAGCCATCTGTTGTTT
GCCCCTCCCCCGTGCCTTCC
TTGACCCTGGAAGGTGCCACTCCCACTGTCCTTTCCTAATAAAATGAGGA
AATTGCATCGCATTGTCTGA
GTAGGTGTCATTCTATTCTGGGGGGTGGGGTGGGGCAGGACAGCAAGGGG
GAGGATTGGGAAGACAATAG
CAGGCATGCTGGGGATGCGGTGGGCTCTATGGCTTCTGAGGCGGAAAGAA
CCAGCTGGGG

In the context of the vector of the invention, such regulatory sequences will be cis-acting. However, the invention also encompasses the use of trans-acting regulatory sequences located on additional genetic constructs.

A preferred post-regulatory element for use in a vector of the invention is the woodchuck hepatitis postregulatory element (WPRE) or a variant thereof. An example sequence of the WPRE is shown below:

(SEQ ID NO: 6)
ATCAACCTCTGGATTACAAAATTTGTGAAAGATTGACTGGTATTCTTAA
CTATGTTGCTCCTTTTACGCT
ATGTGGATACGCTGCTTTAATGCCTTTGTATCATGCTATTGCTTCCCGT
ATGGCTTTCATTTTCTCCTCC
TTGTATAAATCCTGGTTGCTGTCTCTTTATGAGGAGTTGTGGCCCGTTG
TCAGGCAACGTGGCGTGGTGT
GCACTGTGTTTGCTGACGCAACCCCCACTGGTTGGGGCATTGCCACCAC
CTGTCAGCTCCTTTCCGGGAC
TTTCGCTTTCCCCCTCCCTATTGCCACGGCGGAACTCATCGCCGCCTGC
CTTGCCCGCTGCTGGACAGGG
GCTCGGCTGTTGGGCACTGACAATTCCGTGGTGTTGTCGGGGAAATCAT
CGTCCTTTCCTTGGCTGCTCG
CCTGTGTTGCCACCTGGATTCTGCGCGGGACGTCCTTCTGCTACGTCCC
TTCGGCCCTCAATCCAGCGGA
CCTTCCTTCCCGCGGCCTGCTGCCGGCTCTGCGGCCTCTTCCGCGTCTT
CGCCTTCGCCCTCAGACGAGT
CGGATCTCCCTTTGGGCCGCCTCCCCGC

The invention encompasses the use of any variant sequence of the WPRE which increases expression of the transgene compared to a vector without a WPRE. Preferably, variant sequences display at least 70% homology to SEQ ID NO: 6 over its entire sequence, more preferably 75%, 80%, 85%, 90% and more preferably at least 95%, 96% 97%, 98% or 99% homology to SEQ ID NO: 6 over its entire sequence.

Another regulatory sequence which may be used in a vector of the present invention is a scaffold-attachment region (SAR). Additional regulatory sequences may be readily selected by the skilled person.

### Method of Administration

In one embodiment of the present invention, the AAV vector is administered to the eye of a subject by subretinal, direct retinal or intravitreal injection.

The skilled person will be familiar with and well able to carry out individual subretinal, direct retinal or intravitreal injections.

Preferably, the AAV vector is administered by subretinal injection.

### Subretinal Injection

Subretinal injections are injections into the subretinal space, i.e. underneath the neurosensory retina. During a subretinal injection, the injected material is directed into, and creates a space between, the photoreceptor cell and retinal pigment epithelial (RPE) layers.

When the injection is carried out through a small retinotomy, a retinal detachment may be created. The detached, raised layer of the retina that is generated by the injected material is referred to as a “bleb”.

The hole created by the subretinal injection must be sufficiently small that the injected solution does not significantly reflux back into the vitreous cavity after administration. Such reflux would be particularly problematic when a medicament is injected, because the effects of the medicament would be directed away from the target zone. Preferably, the injection creates a self-sealing entry point in the neurosensory retina, i.e. once the injection needle is removed, the hole created by the needle reseals such that very little or substantially no injected material is released through the hole.

To facilitate this process, specialist subretinal injection needles are commercially available (e.g. DORC 41G Teflon subretinal injection needle, Dutch Ophthalmic Research Center International BV, Zuidland, The Netherlands). These are needles designed to carry out subretinal injections.

Unless damage to the retina occurs during the injection, and as long as a sufficiently small needle is used, substantially all injected material remains localised between the detached neurosensory retina and the RPE at the site of the localised retinal detachment (i.e. does not reflux into the vitreous cavity). Indeed, the typical persistence of the bleb over a short time frame indicates that there is usually little escape of the injected material into the vitreous. The bleb may dissipate over a longer time frame as the injected material is absorbed.

Visualisations of the eye, in particular the retina, for example using optical coherence tomography, may be made pre-operatively.

### Two-Step Subretinal Injection

The vector of the present invention may be delivered with increased accuracy and safety by using a two-step method in which a localised retinal detachment is created by the subretinal injection of a first solution. The first solution does not comprise the vector. A second subretinal injection is then used to deliver the medicament comprising the vector into the subretinal fluid of the bleb created by the first subretinal injection. Because the injection delivering the medicament is not being used to detach the retina, a specific volume of solution may be injected in this second step.

In one embodiment of the present invention, the AAV vector is delivered by:

(a) administering a solution to the subject by subretinal injection in an amount effective to at least partially detach the retina to form a subretinal bleb, wherein the solution does not comprise the vector; and

(b) administering a medicament composition by subretinal injection into the bleb formed by step (a), wherein the medicament comprises the vector.

The volume of solution injected in step (a) to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the medicament composition injected in step (b) may be, for example, about 10-500 μL, for example about 50-500, 100-500, 200-500, 300-500, 400-500, 50-250, 100-250, 200-250 or 50-150 μL. The volume may be, for example, about 10, 50, 100, 150, 200, 250, 300, 350, 400, 450 or 500 μL. Preferably, the volume of the medicament composition injected in step (b) is 100 μL. Larger volumes may increase the risk of stretching the retina, while smaller volumes may be difficult to see.

The solution that does not comprise the medicament (i.e. the “first solution” of step (a)) may be similarly formulated to the solution that does comprise the medicament, as described below. A preferred solution that does not comprise the medicament is balanced saline solution (BSS) or a similar buffer solution matched to the pH and osmolality of the subretinal space.

### Visualising the Retina During Surgery

Under certain circumstances, for example during end-stage retinal degenerations, identifying the retina is difficult because it is thin, transparent and difficult to see against the disrupted and heavily pigmented epithelium on which it sits. The use of a blue vital dye (e.g. Brilliant Peel®, Geuder; MembraneBlue-Dual®, Dorc) may facilitate the identification of the retinal hole made for the retinal detachment procedure (i.e. step (a) in the two-step subretinal injection method of the present invention) so that the medicament can be administered through the same hole without the risk of reflux back into the vitreous cavity.

The use of the blue vital dye also identifies any regions of the retina where there is a thickened internal limiting membrane or epiretinal membrane, as injection through either of these structures would hinder clean access into the subretinal space. Furthermore, contraction of either of these structures in the immediate post-operative period could lead to stretching of the retinal entry hole, which could lead to reflux of the medicament into the vitreous cavity.

### Pharmaceutical Compositions and Injected Solutions

The medicaments, for example vectors, of the present invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, e.g. subretinal, direct retinal or intravitreal injection.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

### Method of Treatment

It is to be appreciated that all references herein to treatment include curative, palliative and prophylactic treatment; although in the context of the present invention references to preventing are more commonly associated with prophylactic treatment. Treatment may also include arresting progression in the severity of a disease.

The treatment of mammals, particularly humans, is preferred. However, both human and veterinary treatments are within the scope of the present invention.

### Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” of full length CNTF are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### EXAMPLES

### Example 1

### Materials and Methods

### Mice and Anaesthesia

All animal experiments were performed in compliance with the ARVO statement for the Use of Animals in Ophthalmic and Vision Research and were conducted under a valid UK Home Office licence. Rhodopsin knockout mice (C57B/6.129 Rho^{tmlPhm}, referred to herein as Rho^{-/-}) have been described previously (Humphries, M. M. et al. (1997) Nat. Genet. 15: 216-219). and were obtained as a gift from Jane Farrar, Trinity College Dublin, Ireland. OPN1LW-EGFP mice (C57BL/6J^{TgOPN1LW-EGFP85933Hue} referred to herein as B6^{TgOPN1LW-EGFP}) have been described previously (Fei, Y. et al. (2001) Vis. Neurosci. 18: 615-623) and were obtained from the Mutant Mouse Regional Resource Centres (MMRRC), National Institute of Health, USA, with the kind help of Dr Rachel Pearson, University College London (UCL) Institute of Ophthalmology, London, UK. Mice that express EGFP in cone photoreceptors and have a primary rod specific degeneration (Rho^{-/-TgOPN1LW-EGFP+/-}) were created through crossing of B6^{TgOPN1LW-EGFP+/+} mice (homozygous for the OPN1LW-EGFP transgene insertion) with Rho^{-/-} (homozygous for a targeted rhodopsin knockout), followed by backcrossing of F1 progeny (Rho^{+/-TgOPN1LW-EGFP+/-}) to the parental Rho^{-/-} line. Mice were phenotyped at weaning by AF imaging, and genotyped by PCR. All mice were housed under standard 12 h:12 h light/dark cycle with food and water available ad libitum. General anaesthesia was induced by a single intraperitoneal injection of Dormitor (medetomidine hydrochloride, 1 mg/kg body weight) and ketamine (60 mg/kg body weight) and the pupils fully dilated with 1% tropicamide and 2.5% phenylephrine hydrochloride eye drops (both Bausch & Lomb, Kingston-Upon-Thames, UK). Where appropriate, anaesthesia was reversed following procedures by intraperitoneal injection of antisedan (atipamezole, 5 mg/kg body weight).

### Vector Construction

An Adeno-associated virus serotype 2 vector backbone plasmid (pUF11), was provided by William W. Hauswirth, University of Florida, USA. cDNA clones of human neuronal growth factor (NGF; NM_002506) and human ciliary neurotrophic factor (CNTF; NM_000614) were purchased from Origene (Rockville, Md., USA). PCR was used to amplify the NGF secretion signal (sense primer, GTACGCGGCCGCATGTCCATGTTGTTC; antisense primer, GCTCTGTGAAAGCTGAGTGTGGTTCC) and the CNTF coding region minus the translation start codon and surrounding sequence with identity to the kozak consensus sequence (sense primer, GGAACCACACTCAGCTTTCACAGAGC; antisense primer, TCTAGTCGACCTACATTTTCTTG) from the respective constructs. The two fragments were subsequently combined using a “swift PCR for ligating in vitro constructed exons” (SPLICE) reaction (Davies, W. L. et al. (2007) Biotechniques 43: 785-789) and ligated into the pUF11 backbone using synthetic NotI and AccI restriction sites (underlined). Fidelity of the final AAV2.CBA.hCNTF construct was confirmed by full-length sequencing in both orientations.

### Virus Production and Titration

Recombinant AAV serotype 2 (rAAV2/2) virus packaging the CNTF construct (termed rAAV2/2.hCNTF) was produced by transient co-transfection of HEK293 cells seeded in cell factories (HYPERflask; Corning, Tewksbury, Mass., USA) followed by purification using iodixanol gradient centrifugation, as previously reported (Zolotukhin, S. (2005) Hum. Gene Ther. 16: 551-557; Jacobson, S. G. et al. (2006) Mol. Ther. 13: 1074-1084). Purified rAAV2/2.hCNTF virus was concentrated by buffer exchange (Amicon Ultra-15, Millipore, Billerica, Mass., USA) that removed residual iodixanol before being resuspended in sterile phosphate buffered saline (PBS) to a total volume of 100 μL and aliquoted into pre-blocked tubes (0.01% BSA). The virus was titrated by quantitative PCR (qPCR) using primers designed to amplify a 120 bp fragment within the poly-A region, using vector plasmid and virus of known titre as standards. The titre of rAAV2/2.hCNTF virus was calculated to be 2×10¹³ genome particles (gp) per mL.

### Intraocular Injection

The pupils of four week old Rho^{-/-TgOPN1LW-EGFP+/-} mice were dilated as above and a gel lubricant (Viscotears, Novartis, Frimley, UK) was applied prior to the positioning of a 6 mm circular cover slip over the cornea to allow good visualisation of the retina when viewed under a surgical microscope (M620 F20, Leica, Wetzlar, Germany). Injections were performed by advancing a Hamilton syringe with a 10 mm 34-gauge needle (65N, Hamilton AG) trans-sclerally through the neural retina into the vitreous. 2 μL of vector suspension or buffer (PBS) was delivered into the vitreous cavity close to the posterior pole; reflux was minimized by allowing the intraocular pressure to normalise prior removing the needle. Orientation and stabilisation of the eye was maintained throughout by holding the superior rectus muscle with notched forceps.

### Enzyme Linked Immunosorbent Assays (ELISA)

HEK293 cells were seeded in 6-well plates (1×10⁶ cells per well) and transduced with 2 μL rAAV2/2.hCNTF (1×10¹³ gp/mL) virus. Three days post transduction media was harvested and assayed using a commercially available human CNTF-specific ELISA (R&D systems, Abingdon, UK) according to the manufacturer's instructions. In vivo assessment of hCNTF expression was carried out by intravitreal injection of postnatal week four (PW4) Rho^{-/-TgOPN1LW-EGFP+/-} mice with 2×10⁸ gp (low dose), 2×10⁹ gp (medium dose) or 2×10¹⁰ gp (high dose) rAAV2/2.hCNTF vector. Four weeks were allowed for genome integration and vector expression at which time (PW8) eyes were harvested and flash frozen with liquid nitrogen. Globes were thawed in 100 μL PBS and homogenised prior to hCNTF protein levels being assessed using a commercially available human CNTF-specific ELISA (R&D systems, Abingdon, UK) as stated above.

### Autofluorescence (AF) Imaging and Cone Quantification

The ocular fundi of each mouse were imaged using a confocal scanning laser ophthalmoscope (cSLO; Spectralis HRA, Heidelberg Engineering, Heidelberg, Germany) at weeks 8, 10, 12, 15, 18, 21, 24 and 30, as previously described (Charbel Issa, P. et al. (2011) Optimization of in vivo confocal autofluorescence imaging of the ocular fundus in mice and its application to models of human retinal degeneration. Invest. Ophthalmol. Vis. Sci.). Briefly, following dilation a contact lens was placed on the cornea using a viscous coupling gel (0.3% w/v hypromellose, Matindale Pharmaceuticals, Romford, UK) to prevent cataract formation and to improve and standardise image quality. The mouse was positioned on an imaging platform and the near-infrared (NIR) reflectance mode (820 nm laser) used to achieve camera alignment at the confocal plane of the neural retina. Intrinsically EGFP expressing cone photoreceptors were imaged using the autofluorescence (AF) mode (480 nm excitation) using a 55° lens and high resolution images (1536×1536 pixels) were recorded at a standardised detector sensitivity with automated real-time (ART) averaging. Cone numbers were quantified manually from each image using ImageJ software (Abramoff, M. D. et al. (2004) Biophotonics International 11: 36-42), using vascular landmarks in the inferior retina to standardise the region of interest for each eye across the time course.

### Electroretinography (ERG)

ERG recording was carried out using an Espion E2 system (Diagnosys LLC, Cambridge, UK) according to a standard protocol at PW8, PW10, and PW12, as described previously (Lipinski, D. M., et al. (2011) Invest. Ophthalmol. Vis. Sci. 52: 6617-6623). Following a period of light adaptation (>10 min, polychromatic white light, 30 cd/m²) brief (4 ms) white flashes were delivered at 3, 10 and 25 cd·s/m² (20 flashes per intensity) and the b-wave amplitudes quantified with the Espion software (Diagnosys LLC, Cambridge, UK).

### Optomotor Response (OMR)

A custom built optomotor system was produced consisting of a rotating cylinder which allowed speed and grating size to be precisely regulated. Mice were placed on a raised platform in the centre of the drum which was illuminated from above by a bright white stimulus (˜1000 lux) and acclimatised to the drum for 1 min during which the drum remained stationary and the mice were free to explore the platform. Each experimental run consisted of a 1 min clockwise rotation and a 1 min anti-clockwise rotation divided into alternating 30 s periods. The experimental run was repeated independently three times at each time point for each animal, and the number of responses was averaged. The rotation of the square-wave grating elicited head-tracking responses, with a single response consisting of a slow head-tracking motion in the direction of the drum's rotation followed by a rapid repositioning of the head to a central position. The behaviour of each mouse in response to the rotating drum was recorded using a digital camera mounted directly above the central platform; the number of head-tracking responses was quantified manually with the scorer blinded with regards to treatment.

### Laser Speckle Cortical Imaging

Cortices were imaged of 30 week old Rho^{-/-TgOPN1LW-EGFP+/-} mice that had received rAAV2/2.hCNTF treatment in one eye and PBS sham treatment in the contralateral eye (n=4, high dose; n=6, medium dose; n=8 low dose). The positive control group consisted of mice (n=3) of identical background at an age (6 weeks old) when cone degeneration had not yet commenced. Mice were dark adapted for 8 h prior to imaging and all preparatory steps conducted in a dark room under dim red illumination. Mice were anaesthetised and the pupils fully dilated as described above. The head was secured in a stereotactic frame, and the scalp was resected to reveal the cranium over the visual cortices. Transcranial imaging was performed using the Speckle Contrast Imager (moorFLIP, Moor instruments, Delaware, USA) and data were acquired at 25 Hz. Core body temperature was monitored by rectal thermometry and maintained throughout by means of a feedback heat pad wrapped around the body of the experimental animal. Changes in cortical blood flow (CBF) were measured following the stimulation of each eye independently, where the contralateral eye was covered throughout to maintain dark adaptation. The eye to be examined first was randomised for each mouse and the investigators blinded as to which eye had received rAAV.hCNTF treatment. Each eye was stimulated by brief (10 ms) flashes of 14.5 cd 510±3 nm monochromatic light at 5 Hz for 1 s (Grass PS33 photic stimulator with LED stimulus). Stimulation was repeated 10 times per eye with an interstimulus interval of 30 seconds (5 minute total time). The light stimulus was immediately transferred to the contralateral eye for examination, during which time the mouse and imaging equipment remained static. Data were processed using MATLAB R2012b (version 8.0.0.783). After down-sampling the data to 5 Hz, regions of interest (ROIs) were selected for the bilateral visual cortices from which the time-series changes where extracted and averaged over the pixels within each ROI. Individual time-series were then smoothed using a Chebyshev type 1 filter. The percentage change in CBF relative to a 10 s prestimulus baseline period was calculated for left and right hemispheres. The responses of both hemispheres were summed (contralateral+ipsilateral) for each eye examination, and the magnitude change in CBF compared between rAAV.hCNTF-treated and PBS sham-treated eyes.

### RNA Sequencing and Analysis

Following euthanasia, murine eyes were enucleated and snap frozen in RNAlater (Ambion, Paisley, UK) on liquid nitrogen and stored at −80° C. until processing. Whole eyes were homogenised and total RNA was extracted (RNA tissue mini kit, QIAGEN, Manchester, UK). Contaminating DNA was removed by DNase I treatment following an “on column” digestion protocol (RNase-Free DNase, QIAGEN, Manchester, UK). Poly-adenylated mRNA was purified from 2 μg total RNA per sample by magnetic isolation (NEBNextPoly(A) mRNA magnetic isolation module, New England Biolabs, Hitchin, UK). A library preparation protocol was performed using NEBNext mRNA Library Prep Master Mix Set for Illumina Kit (New England Biolabs, Hitchin, UK) with the following modifications: Following fragmentation RNA underwent additional clean up (Ampure XP, 2.8× volume ratio, Ambion, Paisley, UK) and washing (2×80% ethanol) steps prior to elution in EB buffer. Reverse transcription (RT) was carried out with Superscript II (Invitrogen, Paisley, UK) with post RT clean up (Ampure XP, 1.2× volume ration) and wash (2×80% ethanol) steps. cDNA was end-repaired, A-tailed and adapter ligated (custom adapter) prior to PCR amplification (12 cycles). Prepared libraries were multiplexed (4×) and assessed for quality prior to paired-end sequencing on the IlluminaHiSeq 2000 platform with read length of 50 bp. Output FASTQ files were assessed for quality (SAMStat and FastQC:Read QC), groomed (FASTQ groomer; Blankenberg, D. et al. (2010) Bioinformatics 26: 1783-1785) prior to adapter removal (Cutadapt) and aligned to the reference genome (Mus musculus, UCSC, mm9.fa) in a paired-end manner (TopHat for Illumina; Trapnell, C. et al. (2009) Bioinformatics 25: 1105-1111). Output of accepted hits was converted to SAM format (SAM Tools) and hits assigned to genomic features by comparison to the reference genome with HiSeq-Count. Differential expression was called using the DESeq R package (Bioconductor) with normalisation for effective library size and false discovery rate (FDR) set at 2%. Differentially expressed genes were considered as those with minimum 2-fold (1 2 log-fold) change in expression level. Heatmap outputs were created with RColorBrewer and gplots packages for R.

### Statistics

Anderson-Darling tests for normality on full data sets for each experiment revealed that the data was sampled from a normally distributed population in the following cases: In vivo cone quantification, A²=0.509, p=0.1983; low dose ERG, A²=0.3402, p=0.4824; medium dose ERG, A²=0.4483, p=0.2785; high dose ERG, A²=0.7228, p=0.0594. Plotting of residuals versus fitted means for each experimental group demonstrated that the variance was equal within each group (plots not shown). Repetitive measures two-way ANOVA was used to analysis in vivo cone quantification with dose (independent variable) and time (repeated measure) as factors, and cone number as the dependent variable. Repetitive measures two-way ANOVA was used to analyse ERG responses with flash intensity (independent variable) and time (repetitive measure) as factors, with photopic b-wave amplitude the dependent variable in each case, where each dose was analysed independently. Normality was not confirmed for optomotor response data: A²=4.5536, p=<0.0005. Non-parametric Kruskal-Wallis test with Dunn's multiple comparisons test were performed to compare mean head tracking responses elicited from treated and untreated eyes at each dose. Due to low sample size variance could not be plotted for groups when assessing CNTF levels following in vitro or in vivo vector transduction. Nonparametric Mann-Whitney U tests were applied in both instances to compare mean CNTF levels in transduced versus sham transduced cell supernatants (in vitro) or harvested eyes (in vivo). Bonferroni post tests were applied to all ANOVAs. The significance (p) level for all tests was set at 0.05. Controls. ns=not significant, ***=p<0.001, one way ANOVA. Low dose n=5, medium dose n=6, high dose n=4.

### Results

To model accurately cone survival and function in response to long-term CNTF therapy, C57B/6.129 Rho^{tm1Phm} mice homozygous for knockout of rhodopsin (Rho) were crossed with C57BL/6J^{TgOPN1LW-EGFP85933Hue} transgenic mice that express enhanced green fluorescent protein (EGFP) in cone photoreceptors (Fei, Y. et al. (2001) Vis. Neurosci. 18: 615-623; Humphries, M. M. et al. (1997) Nat. Genet. 15: 216-219). In this model of end-stage retinitis pigmentosa (RP; termed herein, Rho^{-/-TgOPN1LW-EGFP+/-}) apoptosis of rod photoreceptors due to the absence of rhodopsin protein occurs by postnatal week (PW) eight and is followed by the secondary loss of intrinsically fluorescent cones, leading to total photoreceptor degeneration (Hobson, A. H. et al. (2000) Exp. Eye Res. 71: 247-254). The utilisation of an animal model whereby cone photoreceptors degenerate secondary to advanced rod loss reflects the disease phenotype most commonly observed in RP patients (rod-cone dystrophy). Furthermore, the model closely recapitulates the clinical scenario in which neuroprotection would most likely be applied, where the majority of rod photoreceptors have degenerated and the priority is to preserve cone mediated vision irrespective of the etiology underlying photoreceptor loss. This approach is particularly relevant to RP, where the cause of photoreceptor loss is incompletely understood in 50% of autosomal dominant and 30% of autosomal recessive cases (Hartong, D. T. et al. (2006) Lancet 368: 1795-1809; Lipinski, D. M. et al. (2013) Prog. Retin. Eye Res. 32: 22-47).

A recombinant adeno-associated virus serotype 2 (rAAV2/2)-based vector was manufactured (1×10¹³ genome particles (gp) per mL) to express human CNTF (hCNTF) protein modified for extracellular release through addition of an upstream human nerve growth factor secretion signal. Protein secretion from the resultant rAAV2/2.hCNTF vector was validated in vitro by the transduction of HEK293 cells (MOI=1000), resulting in high levels of hCNTF protein being detectable in the media by enzyme-linked immunosorbent assay (ELISA; FIG. 6a). In vivo administration of rAAV2/2.hCNTF into the vitreous cavity (intravitreal injection) of PW4 Rho^{-/-TgOPN1LW-EGFP+/-} mice (n=3 per group) similarly resulted in high levels of secreted hCNTF protein, most likely from ganglion and Müller glia cells which are known to be transduced efficiently by AAV2-based vectors. Importantly, we observed that altering the number of genome particles administered per eye could broadly control the effective dose of hCNTF protein (FIG. 6b).

### CNTF Prevents Development of RP in a Dose Dependent Manner

Rho^{-/-TgOPN1LW-EGFP+/-} mice received an intravitreal injection of rAAV2/2.hCNTF at low (2×10⁸ gp; n=8 mice), medium (2×10⁹ gp; n=6 mice) or high (2×10¹⁰ gp; n=5 mice) dose prior to significant cone photoreceptor loss (PW4). The contralateral eye in all mice received an equivalent volume (2 μL) sham injection of phosphate buffered saline (PBS) to control for the effects of the surgical intervention.

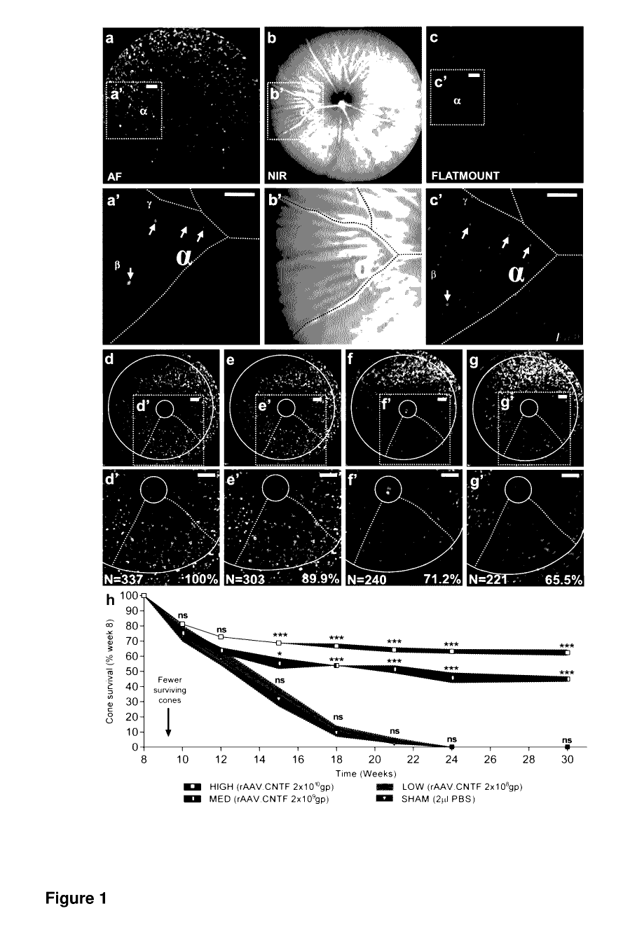

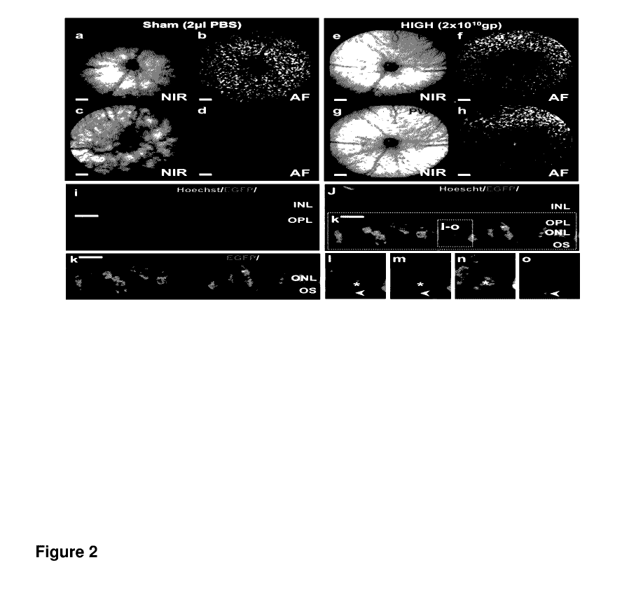

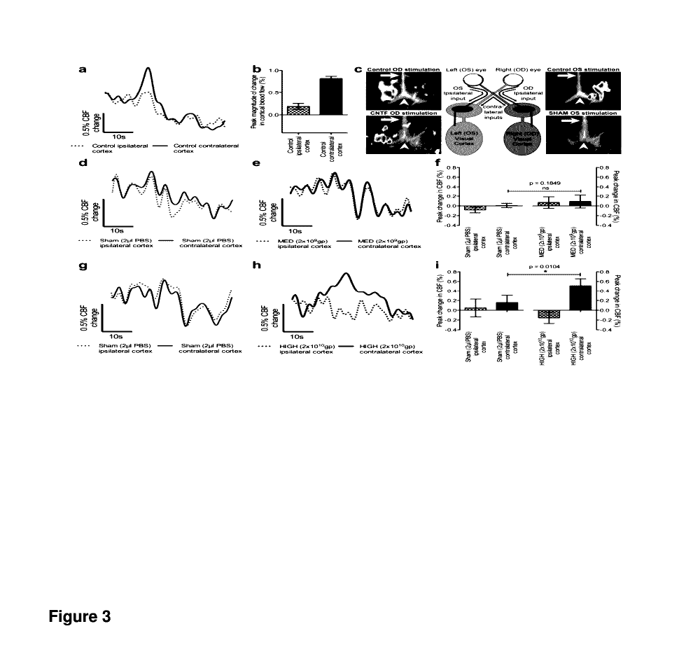

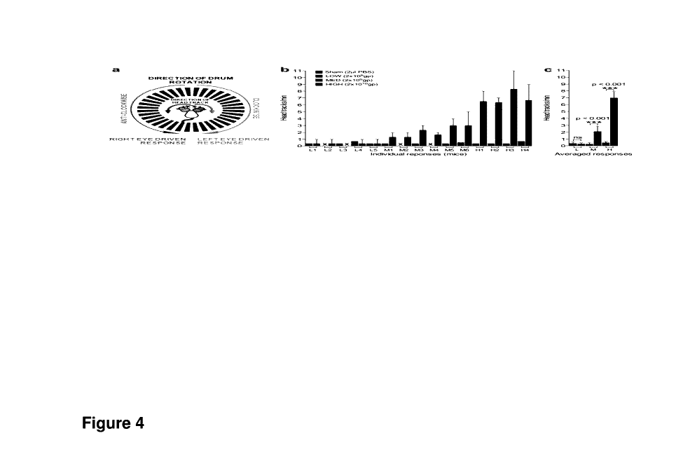

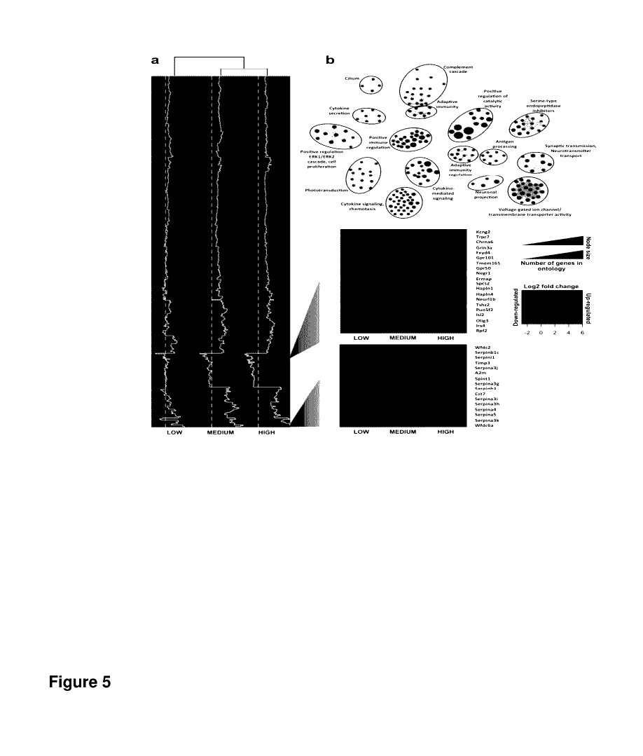

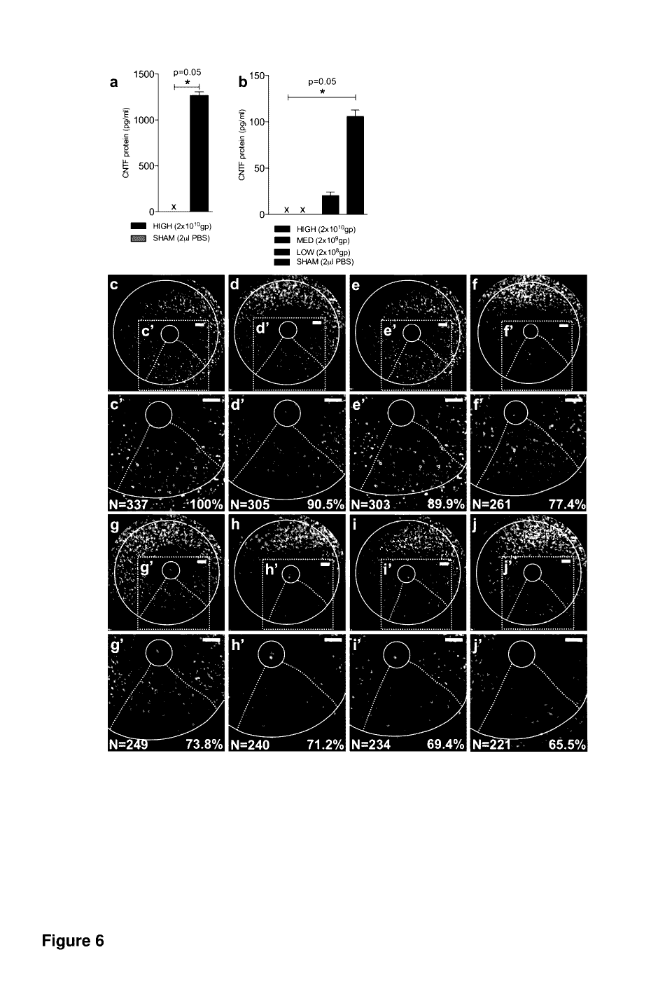

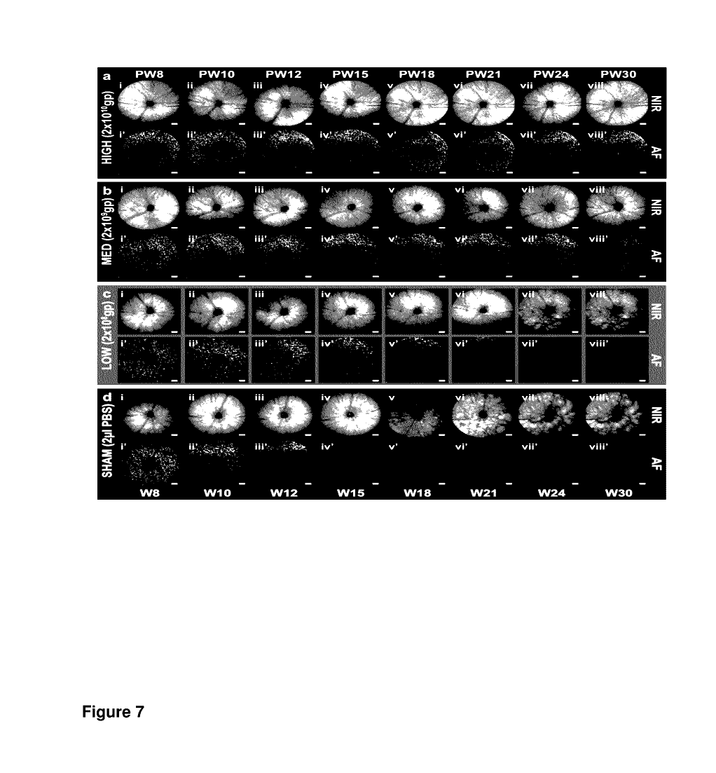

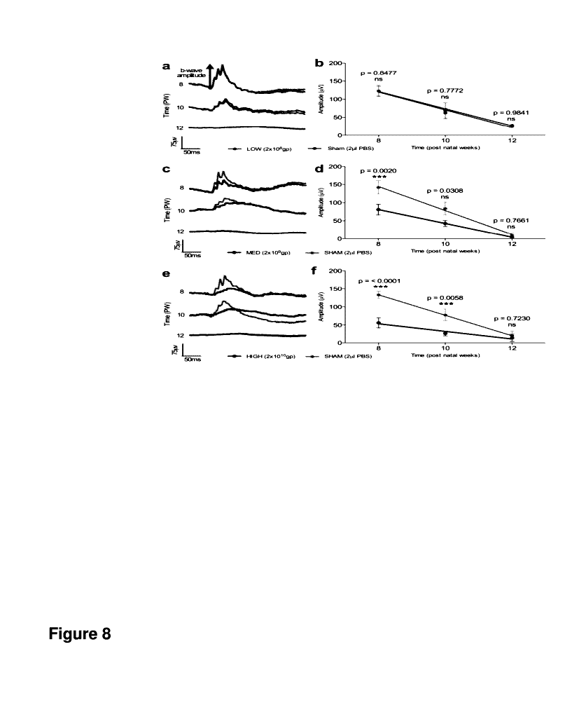

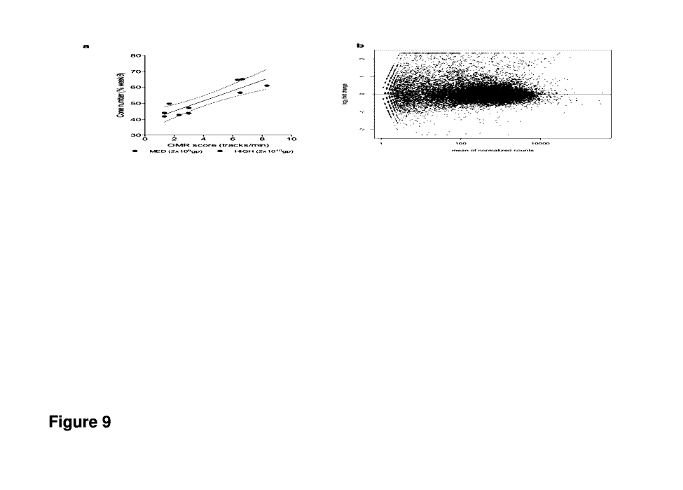

Four weeks post injection (PW8) the retinae of injected mice were imaged with a confocal scanning laser ophthalmoscope (cSLO), which allows non-invasive assessment of retinal pathology by direct visualisation of the fundus through the pupil. Specifically, the cSLO autofluorescence (AF) imaging mode utilises a laser with an excitation wavelength proximate to the absorption peak of EGFP, allowing the visualisation of EGFP-expressing cells within the retina (Beck, S. C. et al. (2010) Invest. Ophthalmol. Vis. Sci. 51: 493-497; Charbel Issa, P. et al. (2011) Optimization of in vivo confocal autofluorescence imaging of the ocular fundus in mice and its application to models of human retinal degeneration. Invest. Ophthalmol. Vis. Sci.). AF imaging of the Rho^{-/-TgOPN1LW-EGFP+/-} mouse fundus revealed a dot like pattern (FIG. 1a) corresponding to the expected distribution of EGFP-expressing cone photoreceptors across the retina. Post-mortem lectin staining (FIG. 1c) of retinas isolated from Rho^{-/-TgOPN1LW-EGFP+/-} mice immediately following AF imaging confirmed that individual “dot-like” signals observed in vivo corresponded to single EGFP-expressing cone photoreceptors (FIG. 1a-c). Using vascular landmarks to define a standardised region of interest within each eye that would remain constant throughout the life of an experimental animal, it was possible to quantify accurately cone photoreceptor numbers and to assess survival longitudinally in response to hCNTF treatment (FIG. 1d-g; FIG. 6c-j).

AF imaging demonstrated a comparable distribution of EGFP-expressing cone photoreceptors in all treatment and control groups at PW8 (FIG. 1h; FIG. 7a-d). Cone photoreceptor numbers declined rapidly in the PBS sham treated-group over the experimental time course, with cone numbers reaching zero at PW24 (FIG. 1h, FIG. 7d). Consistent with the absence of detectable hCNTF protein (FIG. 6b), cone photoreceptor numbers in eyes that received a low dose (2×10⁸ gp) intravitreal injection of rAAV2/2.hCNTF were not significantly different to paired PBS sham injected eyes at any time point (FIG. 1h; FIG. 7c; p≧0.05, repeated measures two-way ANOVA with Bonferroni post-test, n=8). Near-infrared reflectance (NIR) imaging revealed focal geographic changes in the retinal pigment epithelium (RPE) consistent with loss of the overlying photoreceptors, a common clinical observation in late-stage RP (FIG. 2a-d, FIG. 7c-d). This observation is supported by histology conducted at PW30 that demonstrated the absence of both rod and cone photoreceptors in PBS sham treated eyes (FIG. 2i).

Cone numbers in eyes treated with a medium (2×10⁹ gp) or high (2×10¹⁰ gp) dose of rAAV2/2.hCNTF vector were significantly greater in comparison to contralateral control eyes from PW12 onwards (FIG. 1h; FIG. 7a-b). Neuroprotection of cone photoreceptors was dose dependent, with greatest survival being observed in high dose treated eyes (62.38±1.95%) compared to medium dose-treated eyes (44.90±1.69%; PW30 vs. PW8). The rate of cone photoreceptor loss was near-zero from PW15 onwards in both treatment groups (FIG. 1h; 0.42-0.69%/week); no RP phenotype was observed in medium or high dose-treated eyes (FIG. 2e-h; FIG. 7a-b). Histology at PW30 revealed preservation of both cone (GFP-expressing) and rod (non GFP-expressing) photoreceptors in eyes receiving high dose rAAV2/2.hCNTF vector (FIG. 2j). The absence of EGFP co-localisation with medium wave sensitive (Mws) cone opsin expression strongly indicates the presence of discrete cone outer segments (FIG. 2k-o), where the connecting cilium effectively prohibits the trafficking of EGFP protein from the photoreceptor cell body the outer segments when present.

### CNTF Preserves Visual Function Despite Mild Suppression of Electrophysiological Responses

rAAV2/2.hCNTF treated mice were examined at PW8 by electroretinography (ERG), a technique that utilises a corneal electrode to indirectly measure the massed electrical response of the retina following stimulation with light. In line with evidence of previous studies that demonstrated reduced cone sensitivity in the presence of CNTF protein, cone photoreceptor-mediated responses were observed to be suppressed in a dose-dependent manner (Wen, R. et al (2012) Prog. Retin. Eye Res. 31: 136-151) (FIG. 8a-f). Peak ERG recordings fell below the mean amplitude of background noise (˜20 μV) in all groups by PW12 and were consequently considered to be unrecordable. Whilst the reduction in peak ERG amplitude coincided with a period of significant cone photoreceptor loss in all groups, medium (2×10⁹ gp) and high (2×10¹⁰ gp) dose treated eyes demonstrated significant preservation of cones beyond PW12, potentially indicating the absence of cone-mediated vision despite preservation of cone photoreceptor cell bodies (FIG. 2j-o). We therefore explored visual function in more detail with further electrophysiological and behavioural testing at PW30.

The ability of remaining cones to relay signals through the visual pathway to the brain was assessed directly by performing laser speckle imaging (LSI) of the visual cortex during stimulation of the retina by light. LSI is a non-contact imaging technique with high temporal and spatial resolution that can detect changes in random speckle patterns that are linearly associated with increased movement of red blood cells in the microvasculature (FIG. 3c). Herein, LSI was used to measure changes in the cortical blood flow (CBF) in the visual cortex following monocular stimulation with brief (10 ms) flashes of 14.5 cd monochromatic light (510±3 nm), where the wavelength is proximate to the maximal peak of absorption of the murine M-cone. The ability of the chosen stimulation parameters to elicit a cone-mediated response was validated on PW6 untreated Rho^{-/-TgON1LW-EGFP+/-} mice (n−3). Changes in CBF were detected primarily in the visual cortex contralateral to the eye stimulated, indicating appropriate down-stream processing of the visual stimulus (FIG. 3a-c). Cortical LSI at PW30 during stimulation of eyes treated with a medium dose (2×10⁹ gp) of rAAV2/2.hCNTF vector revealed increased, but not significant, CBF changes in the contralateral visual cortex (FIG. 3d-f; p=0.20, one-tailed Wilcoxon matched-pairs, n=6). By contrast, LSI during stimulation of eyes that received a high dose (2×10¹⁰ gp) of rAAV2/2.hCNTF demonstrated an increased CBF in the contralateral visual cortex that was significantly greater than the response elicited during stimulation of the sham-treated control eyes (FIG. 3c, g-i; p=0.012, one-tailed Wilcoxon matched-pairs, n=4). When compared to baseline LSI CBF measurements obtained from PW6 animals (+0.819%), the magnitude of CBF change elicited following stimulation of high (+0.509%) dose CNTF treated eyes broadly correlates to the percentage cone photoreceptor survival observed at PW30 (62.38±1.95% of PW8). Cortical LSI strongly indicated that surviving cones remained light sensitive and were able to relay signals to the brain. The extent to which changes in cortical blood flow corresponded to the processing of useful vision was assessed by measurement of optomotor response (OMR). OMR measures the number of involuntary head-tracking movements an experimental animal makes in response to a full field visual stimulus, classically consisting of a brightly lit (˜1000 lux) rotating drum with vertically orientated black and white stripes of a defined width and contrast (100% contrast; 0.2 cycles/degree) into which the animal is placed (FIG. 4a). OMR is characterised by a slow-phase head-tracking motion in the direction of the drum's rotation, followed by a rapid-phase reorientation of the head to a central position. The slow phase head-tracking movement is driven by each eye independently and relies on the direction of the drum's rotation (FIG. 4a) (Hobbelen, J. F. et al. (1971) Doc. Ophthalmol. 30: 227-236; Harvey, R. J. et al. (1997) Vision Res. 37: 1615-1625; Douglas, R. M. et al. (2005) Vis. Neurosci. 22: 677-684). When experimental animals were examined at PW30 the number of head tracking responses per minute recorded from sham treated eyes was not significantly above zero in any group (0.33±0.05/min). The number of head tracking responses elicited by stimulation of medium dose (2.11±0.31/min) and high dose treated eyes (6.92±0.92/min) were significantly greater than paired sham eyes (FIG. 4b-c; p=<0.001, Kruskal-Wallis test with Dunn's multiple comparisons, n=4), whilst linear regression analysis of OMR responses versus residual cone number at PW30 revealed a significant positive correlation (r²=0.80, F=33, p=0.0004) (FIG. 9a).

Collectively these data strongly indicate that despite the apparent suppression of electrophysiological function as assessed by ERG, cone photoreceptors preserved by hCNTF-mediated neuroprotection remain sensitive to light, and are able to usefully relay signals via the optic nerve to central visual pathways.

### CNTF Upregulates Complement Factor Proteins and Inhibitors of Proteolysis

Transcriptome analysis of 23,365 mouse genes was undertaken to determine the mechanisms underlying long-term neuroprotection of cone photoreceptors in response to CNTF. Transcriptomes of treated eyes were compared at each dose to paired sham treated controls (n=4 eyes per group) by next-generation sequencing of total mRNA extracted from whole eyes at PW30. The false discovery rate was fixed at 2% following the Benjamini-Hochberg procedure and all samples were normalised with respect to the effective library size. When comparing the transcriptomes of high dose treated (2×10¹⁰ gp) eyes with paired PBS sham-treated controls, 1,533 genes were found to show significant differential expression (p<0.05; FIG. 9b). Of those, 460 genes demonstrated a>2-fold change in their expression levels and were subsequently grouped based by cellular function using gene ontology (FIG. 5a-b). Serine-, cysteine- and metallo-type peptidase inhibitors (17 genes; Table 1.1) comprised the most transcriptionally up-regulated family, with members demonstrating up to 89-fold greater expression in high dose hCNTF-treated eyes compared to paired sham-treated controls. Members of this group encode intracellular and extracellular peptidase inhibitors, dysfunction of which have previously been associated with the development of retinal disease; such as tissue inhibitor of metalloproteinase 3 (Timp3, +3.49-fold), mutations in which are linked to autosomal dominant Sorsby's fundus dystrophy (Weber, B. H. et al. (1994) Nat. Genet. 8: 352-356). Transcriptional regulation of peptidase inhibitors is not well understood, but activation appears to be signal transducer and activator of transcription 3 (Stat3) mediated (+2.46-fold expression) and require members of the CCAAT/enhancer binding protein (Cebp) family of transcription factors, three of which (Cebpα, Cebpβ and Cebpδ) were also significantly up-regulated (Table 1.2) (Zhao, H. et al. (2007) Mol. Cell Biol. 27: 5286-5295; Udofa, E. A. et al. (2013) PLoS One 8: e57855).

Several members of the classical and alternative complement cascades (16 genes, Table 1.3) were significantly up regulated (up to 10-fold) including various complement component (e.g. C3, C4a) and complement factor genes (e.g. Cfb). Numerous modulators of cytokine signalling were upregulated (19 genes, up to 8-fold; Table 1.4), including suppressors of cytokine signaling 3 (SOCS3) and 5 (SOCS5), which are known to be expressed in photoreceptors and reduce stress-induced inflammatory responses (Ozawa, Y. et al. (2008) J. Biol. Chem. 283: 24561-24570; Takase, H. et al. (2005) J. Neuroimmunology 168: 118-127).

Down-regulation (1.7-fold) of Lecithin Retinol Acyltransferase (LRAT), a critical component of the vitamin-A cycle, and several genes encoding ion channels and G protein-coupled receptors expressed in inner retinal neurons was observed in medium and high dose rAAV2/2.hCNTF treated eyes (Table 1.5) indicating widespread alteration of phototransduction in response to CNTF treatment, in line with previous studies (Wen, R. et al. (2012) Prog. Retin. Eye Res. 31: 136-151).

### Discussion

Through exploitation of the eye's unique optical properties we were able to assess progressive neuronal degeneration in real-time and accurately quantify the survival of intrinsically fluorescent cone photoreceptors through repetitive in vivo imaging. This work demonstrates for the first time the ability of a neurotropic compound to provide life-long protection in a model of degenerative disease.

Our data demonstrate that cone photoreceptors can be preserved by rAAV-mediated hCNTF secretion beyond the point at which total outer-retinal degeneration would be expected without intervention. Stabilisation of cone photoreceptor numbers was observed in medium (44.90±1.69%) and high (62.38±1.95%) dose treatment groups suggesting that protection may be maintained long-term. These findings are significant in light of recent clinical trials utilising encapsulated cell technology to provide sustained CNTF delivery, where photoreceptor preservation has been demonstrated in the short to medium term (up to 5 years), but the likely outcomes of long term neuroprotection remain unknown due to the slow progressive nature of RP in humans (Birch, D. G. et al. Long-term follow-up of patients with retinitis pigmentosa (RP) receiving sustained-release CNTF through intraocular encapsulated cell technology implants. ARVO annual meeting (Seattle, Wash., 2013); Birch, D. G. et al. (2013) Am. J. Ophthalmol. 156: 283-292 e281).

In the present study, cell survival was observed to be dose-dependent, yet so too was physiological suppression of function as assessed by electroretinography (ERG); a dose was not observed at which neuroprotection was achieved without functional suppression, as had been previously proposed (McGill, T. J. et al. (2007) Invest. Ophthalmol. Vis. Sci. 48: 5756-5766). The number of photoreceptors preserved, and the sensitivity of each photoreceptor to light, appears to be directly proportional to the bioavailability of hCNTF protein. This strongly suggests that the mechanisms underlying functional suppression and cell survival may be inextricably linked. Accordingly, suppression of visual function during the early stages of a progressive degenerative disease, when patients might otherwise have good visual acuity, may be outweighed by the potential to extend vision beyond the point at which blindness would have occurred without therapy.

The time point of intervention is another factor that is likely to be important in successfully preserving cone photoreceptors and maintaining function. In the present study we aimed to model closely the clinical scenario whereby a significant proportion of rod photoreceptors are absent at the point of maximum CNTF expression. This was achieved by timing rAAV.hCNTF administration so that the peak of transgene expression would not occur until PW8-10, by which point rod degeneration is well advanced in the rhodopsin knockout mouse model (Humphries, M. M. et al. (1997) Nat. Genet. 15: 216-219). Whilst our findings are promising, it is worth noting that the histology presented herein clearly demonstrates the preservation of rod photoreceptors (non GFP-expressing cells) in addition to cones following CNTF treatment.

Interestingly, despite the early point of intervention, previous preclinical studies uniformly failed to observe functional preservation, suggesting that maintenance of vision may not be possible even when treatment is given prior to the onset of retinal degeneration. In line with those studies, we initially utilised ERG as a primary assessment of residual electrophysiological function, and observed that measurements became unrecordable in all groups by PW12. As ERG is an indirect measurement of retinal function, dependent upon the propagation of an electrical signal of sufficient amplitude and uniformity to be detected by electrodes placed on the corneal surface, the absence of recordable cone ERGs beyond PW12 was thought to not necessarily reflect an absence of functional vision. Indeed, it is quite possible for small areas of the retina to be light sensitive and yet not propagate a detectable signal, a principle best highlighted by studies focusing on photoreceptor transplantation and optogenetic approaches to restore light sensitivity to degenerate retinae, where functional vision can be partly restored and yet ERG responses almost certainly remain absent (Mace, E. et al. (2015) Mol. Ther. 23: 7-16; Gaub, B. M. et al. (2014) Proc. Natl. Acad. Sci. USA 111: E5574-5583; Singh, M. S. et al. (2013) Proc. Natl. Acad. Sci. USA 110: 1101-1106; Francis, P. J. et al. (2013) Translational vision science & technology 2: 4).

A contributing factor to the absence of ERG responses observed in the present study beyond PW12 may be suppression of outer segment formation, as has been noted with rod photoreceptors following CNTF treatment (Wen, R. et al. (2006) J. Neurosci. 26: 13523-13530). Studies into the effects of CNTF on cone outer segment formation are limited; however, we observed the persistence of defined opsin-containing cone outer segments at PW30, strongly indicating that cones may remain light sensitive despite an absent ERG (Li, Y. et al. (2010) PLoS One 5: e9495; Beltran, W. A. et al. (2007) Exp. Eye Res. 84: 753-771; Rhee, K. D. et al. (2013) Proc. Natl. Acad. Sci. USA 110: E4520-4529). To examine the hypothesis that cone photoreceptors retained the ability to relay interpretable signals to the visual cortex and pretectal area of the brain, we performed laser speckle imaging (LSI). LSI during unilateral ocular stimulation proved a robust method by which to assess the light sensitivity of remaining cones, crucially allowing the delineation of responses from treated and untreated eyes separately within each animal. Cortical imaging revealed that stimulation of high dose rAAV.hCNTF treated eyes elicited a significant change in cortical blood flow in the contralateral visual cortex, strongly suggesting that residual cone photoreceptors were receptive to light and remained correctly synapsed. Importantly, the propagation of an optomotor response revealed that the signals received by the visual cortices encoded physiologically relevant information. Head tracking responses were not elicited following stimulation of sham-treated control eyes, supporting the evidence that intrinsically photoreceptive retinal ganglion cells, which are present in the inner retina even in end-stage degeneration, do not contribute to OMR (Cahill, H. et al (2008) PLoS One 3: e2055). The findings are also of particular relevance to clinical trials expressing hCNTF protein using encapsulated cell technologies, as they demonstrate that despite short-term functional suppression, useful vision may still be preserved in end-stage disease and beyond the point at which blindness may have otherwise occurred.

The impact of CNTF dosing appears to be critical not only in the treatment of RP, where photoreceptors are functionally suppressed in line with increased dose, but is an important consideration when evaluating treatments for other neurodegenerative diseases. In particular, past unsuccessful clinical trials aimed at preventing motor neuron loss in ALS demonstrated an absence of therapeutic efficacy and significant incidence of adverse events in patients treated systemically with high doses of CNTF (A double-blind placebo-controlled clinical trial of subcutaneous recombinant human ciliary neurotrophic factor (rHCNTF) in amyotrophic lateral sclerosis. ALS CNTF Treatment Study Group. (1996) Neurology 46: 1244-1249; Bongioanni, P. et al. (2004) The Cochrane database of systematic reviews, CD004302). These findings indicate that a more targeted approach may be required than offered by current neuroprotective strategies, whereby the beneficial mechanisms underlying cell survival are isolated from those resulting in adverse effects. In this study we were able to reveal unique insights into the potential mechanisms of CNTF-mediated neuroprotection following sustained expression, which until now have remained incompletely understood, by conducting a transcriptome analysis of surviving neurons in end-stage retinal disease. A transcriptome analysis of rAAV2/2.hCNTF-treated versus sham-treated eyes revealed several important findings. First, significant overexpression of the Stat3 gene was observed in medium and high dose rAAV2/2.hCNTF-treated eyes, consistent with previous studies showing that CNTF signals via a receptor complex of Cntfr/Lifrβ/Gp130 activity mediated by Stat3 (Rhee, K. D. et al. (2013) Proc. Natl. Acad. Sci. USA 110: E4520-4529; Davis, S. et al. (1993) Science 259: 1736-1739; Kassen, S. C. et al. (2009) Exp. Eye Res. 88: 1051-1064; Peterson, W. M. et al. (2000) J. Neurosci. 20: 4081-4090).

Second, several families of genes encoding inhibitors of proteolysis were robustly overexpressed (up to 89-fold) indicating a key role in hCNTF-mediated neuroprotection. Activation of members of the serine protease inhibitor (Serpin) family (11 genes) in particular are closely linked to Stat3 expression levels and have proved to be anti-apoptotic when overexpressed in tumours (Ahmed, S. T. et al. (2009) Biochem. Biophys. Res. Commun. 378: 821-825). Inhibitors of proteolysis may play a crucial role in retinal disease, including Timp3 and Serpin1b, which act to prevent degradation of extracellular matrix components and Bruch's membrane, respectively, and are implicated in Sorsby's fundus dystrophy and age-related macular degeneration (AMD) (Weber, B. H. et al. (1994) Nat. Genet. 8: 352-356; Chong, N. H. et al. (2005) Am. J. Pathol. 166: 241-251; Sorsby, A. et al. (1949) Br. J. Ophthalmol. 33: 67-97). With the physical degradation of neurons, such as photoreceptors, in neurodegenerative diseases being typically mediated by serine-cysteine proteases, lysosomal proteases and complement mediate lysis, we hypothesis that the overexpression of endogenous inhibitors of proteolysis observed herein may provide a critical defense mechanism against neuronal apoptosis in response to cellular stress. We propose that direct overexpression of secreted proteolysis inhibitors may provide a novel therapeutic avenue for the prevention of neuronal cell death in degenerative diseases. Indeed, utilising a targeted approach aimed at preventing photoreceptor degeneration in RP through direct inhibition of cell body and extracellular matrix degradation may enable photoreceptors to be preserved without physiological suppression of function. In the case of ALS, preventing degeneration of cortical motor neurons directly through inhibition of proteolysis may allow treatment efficacy to be achieved without the significant adverse effects observed in clinical trials when CNTF was delivered systemically at high dose (A double-blind placebo-controlled clinical trial of subcutaneous recombinant human ciliary neurotrophic factor (rHCNTF) in amyotrophic lateral sclerosis. ALS CNTF Treatment Study Group. (1996) Neurology 46: 1244-1249; Bongioanni, P. et al. (2004) The Cochrane database of systematic reviews, CD004302).

Last, a sustained innate immune response characterised by significant upregulation (up to 10-fold) of several members of the classical and alternative complement cascades (16 genes) was observed in medium and high dose-treated eyes, including two complement factors associated with AMD (encoded by the Cfi gene) and retinal light damage (encoded by the Cfd gene) (van de Ven, J. P. et al. (2013) Nat. Genet. 45: 813-817; Rohrer, B. et al. (2007) Invest. Ophthalmol. Vis. Sci. 48: 5282-5289). Interestingly, no complement or cytokine upregulation was observed in low dose-treated eyes at PW30, indicating that the innate immune response ceases once photoreceptor degeneration is complete. Although the role of several upregulated protease inhibitors remains unknown, the expression of genes that encode proteolysis inhibitors may also be particularly relevant to modifying innate immune responses occurring in the central nervous system, where various components (e.g. C3, C4 and Cfb proteins) are activated only following proteolytic cleavage. Serpin3k, for example, is known to have strong anti-inflammatory and anti-angiogenic properties, inhibiting tumour necrosis factor alpha (Tnf-α), intracellular adhesion molecule 1 (Icam-1) and vascular endothelial growth factor (Vegf) through blockade of the Wnt pathway (Zhang, B. et al. (2009) Invest. Ophthalmol. Vis. Sci. 50: 3943-3952). In addition to preventing retinal necrotic cell death, Serpin3k also significantly inhibits rod outer segment generation, and thus its upregulation by hCNTF may also contribute to reduced retinal sensitivity (Zhang, B. et al. (2009) Invest. Ophthalmol. Vis. Sci. 50: 3943-3952; Zhang, B. et al. (2010) Proc. Natl. Acad. Sci. USA 107: 6900-6905; Zhang, B. et al. (2008) PLoS One 3: e4077).

In summary, this study has demonstrated that the unique optical properties of the eye allow for neuronal survival in response to a therapeutic intervention to be assessed longitudinally and with great accuracy through repetitive in vivo imaging. Herein, we have demonstrated that sustained hCNTF expression leads to life-long preservation of cone photoreceptors, and that despite limited electrophysiological suppression, useful vision was maintained until the end-stages of degeneration. This study demonstrated that robust cell survival is directly linked to the dose of CNTF available. Transcriptome analyses strongly indicate that Stat3-mediated overexpression of intracellular and extracellular protease inhibitors underlie cone preservation in RP following hCNTF treatment, possibly through direct inhibition of cellular and extracellular matrix degradation. We propose that overexpression of specific endogenous protease inhibitors may provide a novel therapeutic avenue for the protection of neurons against cell death in RP/AMD and other neurodegenerative disorders, such as ALS.

TABLE 1.1
Peptidase inhibitors

| Gene | | | | | |
|---|---|---|---|---|---|
| symbol | Description | fold Change | log2 Fold | p-value | adj. p-value |
| Wfdc6a | WAP four-disulphide core domain 6A | 89.34 | 6.48 | 6.20E−72 | 1.57E−69 |
| Serpina3k | Serine/cysteine peptidase inhibitor, clade A, member 3K | 55.43 | 5.79 | 4.77E−34 | 5.17E−32 |
| Serpina5 | Serpin peptidase inhibitor, alpha-1 antitrypsin, antiproteinase), clade A, Member 5 | 34.59 | 5.11 | 1.50E−127 | 2.28E−124 |
| Serpina4 | Serpin peptidase inhibitor, alpha-1 antitrypsin, antiproteinase), clade A, Member 4 | 18.60 | 4.22 | 3.22E−24 | 1.39E−22 |
| Serpina3h | Serine/cysteine peptidase inhibitor, clade A, member 3H | 17.38 | 4.12 | 2.69E−13 | 3.85E−12 |
| Serpina3i | Serine/cysteine peptidase inhibitor, clade A, member 3I | 10.67 | 3.42 | 4.55E−07 | 1.94E−06 |
| Cst7 | Cystatin F (leukocystatin) | 9.90 | 3.31 | 3.89E−22 | 1.31E−20 |
| Serpinh1 | Serpin peptidase inhibitor, clade H (heat shock proetin 47), Member1 | 9.24 | 3.21 | 1.77E−12 | 2.35E−11 |
| Serpina3g | Serine/cysteine peptidase inhibitor, clade A, member 3G | 8.55 | 3.10 | 1.02E−19 | 3.04E−18 |
| Spint1 | Serine peptidase inhibitor, Kunitz type 1 | 6.74 | 2.75 | 4.05E−05 | 9.60E−05 |
| A2m | alpha-2-macroglobulin | 5.28 | 2.40 | 2.31E−33 | 2.34E−31 |
| Serpina3j | Serine/cysteine peptidase inhibitor, clade A (antiprotinase, antitrypsin), member 3J | 4.66 | 2.22 | 1.56E−03 | 2.72E−03 |
| Timp3 | TIMP metallopeptidase inhibitor 3 | 3.49 | 1.80 | 5.45E−16 | 1.03E−14 |
| Serpine3 | serpin peptidase inhibitor, clade E, member 3 | 2.44 | 1.28 | 5.70E−09 | 4.04E−08 |
| Serpini1 | serpin peptidase inhibitor, clade I (neuroserpin), member 1 | 2.39 | 1.25 | 3.90E−29 | 2.19E−27 |
| Serpinb1c | Serine (or cysteine) peptidase inhibitor, clade B, member 1C | 2.22 | 1.15 | 1.14E−04 | 2.38E−04 |
| Wfdc2 | WAP four-disulfide core domain 2 | 2.10 | 1.07 | 9.24E−05 | 1.99E−04 |

TABLE 1.2
Transcription factors

| Gene | | | | | |
|---|---|---|---|---|---|
| symbol | Description | fold Change | log2 Fold C [symbol] | p-value | adj. p-value |
| Pou2f2 | POU class 2 homeobox 2 | 27.05 | 4.76 | 2.52E−05 | 6.22E−05 |
| Nr2f2 | Nuclear receptor subfamily 2, group F, member 2 | 5.90 | 2.56 | 2.39E−09 | 1.85E−08 |
| Bcl3 | B-cell CLL/lymphoma 3 | 4.38 | 2.13 | 9.99E−35 | 1.17E−32 |
| Sin3a | Sin3 transcription regulator homolog A (yeast) | 3.45 | 1.79 | 1.86E−04 | 3.71E−04 |
| Cebpd | CCAAT/enhancer binding protein (C/EBP) | 3.30 | 1.72 | 1.04E−29 | 6.30E−28 |
| E2f8 | E2F transcription factor 8 | 2.67 | 1.42 | 3.01E−05 | 7.27E−05 |
| Sp2 | Sp2 transcription factor | 2.59 | 1.37 | 3.73E−07 | 1.61E−06 |
| Myog | myogenin (myogenic factor 4) | 2.58 | 1.37 | 2.22E−06 | 7.32E−06 |
| Ppargc1b | peroxisome proliferator-activated receptor gamma, coactivator 1 beta | 2.51 | 1.33 | 1.40E−05 | 3.67E−05 |
| Stat3 | Signal transducer and activator of transcription 3 (acute phase response factor) | 2.47 | 1.30 | 3.03E−05 | 7.29E−05 |
| Ncoa2 | nuclear receptor coactivator 2 | 2.46 | 1.30 | 1.14E−04 | 2.38E−04 |
| Sp3 | Sp3 transcription factor | 2.33 | 1.22 | 7.65E−05 | 1.69E−04 |
| Cebpb | CCAAT/enhancer binding protein (C/EBP), beta | 2.30 | 1.20 | 3.47E−17 | 7.87E−16 |
| Sox13 | SRY (sex determining region Y)-box 13 | 2.31 | 1.21 | 8.24E−07 | 3.15E−06 |
| Trim66 | Tripartite motif containing 66 | 2.22 | 1.15 | 1.13E−04 | 2.37E−04 |
| E2f2 | E2F transcription factor 2 | 2.21 | 1.15 | 2.94E−04 | 5.65E−04 |
| Twist2 | Twist basic helix-loop-helix transcription factor 2 | 2.20 | 1.14 | 7.84E−05 | 1.72E−04 |
| Stat5a | signal transducer and activator of transcription 5A | 2.09 | 1.07 | 1.08E−21 | 3.50E−20 |
| Cebpa | CCAAT/enhancer binding protein (C/EBP), alpha | 2.04 | 1.03 | 5.25E−09 | 3.76E−08 |
| Glis3 | GLIS family zinc finger 3 | 2.03 | 1.02 | 1.09E−04 | 2.29E−04 |
| Irx4 | iroquois related homeobox 4 (drosophila) | 0.49 | −1.02 | 4.49E−04 | 8.32E−04 |
| Isl2 | insulin related protein 2 (islet 2) | 0.46 | −1.12 | 8.20E−04 | 1.47E−03 |
| Tfap4 | transcription factor AP-4 (activating enhancer binding protein 4) | 0.42 | −1.27 | 7.42E−04 | 1.34E−03 |
| Pou5f1 | POU domain, class 5, transcription factor 1 | 0.41 | −1.29 | 5.20E−08 | 2.89E−07 |
| Pou5f2 | POU domain, class 5, transcription factor 2 | 0.40 | −1.31 | 5.50E−03 | 8.82E−03 |
| Olig3 | oligodendrocyte transcription factor 3 | 0.37 | −1.43 | 1.22E−13 | 1.82E−12 |
| Hnf4a | hepatocyte nuclear factor 4, alpha | 0.19 | −2.36 | 1.92E−07 | 9.23E−07 |
| Gsx1 | GS homeobox 1 | 0.18 | −2.49 | 2.40E−03 | 4.06E−03 |
| Tinf2 | TERF1 (TRF1)-interacting nuclear factor 2 | 0.18 | −2.48 | 1.41E−11 | 1.61E−10 |

[symbol]

indicates data missing or illegible when filed

TABLE 1.3
Complement

| Gene | | | | | |
|---|---|---|---|---|---|
| symbol | Description | fold Change | log2 Fold C [symbol] | p-value | adj. p-value |
| Cfi | Complement factor I | 10.54 | 3.40 | 3.38E−95 | 1.28E−92 |
| Cfd | Complement factor D (adipsin) | 6.39 | 2.68 | 5.09E−06 | 1.52E−05 |
| C6 | Complement component 6 | 6.03 | 2.59 | 1.08E−04 | 2.28E−04 |
| C3 | Complement component 3 | 5.98 | 2.58 | 8.57E−11 | 8.44E−10 |
| C4b | Complement component 4b | 4.14 | 2.05 | 1.75E−73 | 5.32E−71 |
| C4a | Complement component 4a | 4.04 | 2.01 | 3.93E−47 | 5.96E−45 |
| Cfb | Complement factor B | 3.03 | 1.60 | 1.93E−19 | 5.51E−18 |
| C1qa | complement component 1, Q subcomponent, A chain | 2.84 | 1.51 | 3.63E−16 | 7.15E−15 |
| C1qb | complement component 1, Q subcomponent, B chain | 2.58 | 1.37 | 2.23E−13 | 3.25E−12 |
| C1qc | complement component 1, Q subcomponent, C chain | 2.56 | 1.36 | 1.19E−17 | 2.86E−16 |
| C1rb | complement component 1, R subcomponent B | 2.49 | 1.32 | 3.98E−09 | 2.93E−08 |
| C1ra | complement component 1, R subcomponent A | 2.35 | 1.23 | 1.43E−18 | 3.73E−17 |
| C1rl | Complement component 1, R subcomponent-like | 2.27 | 1.18 | 1.06E−06 | 3.89E−06 |
| C1s | complement component 1, S subcomponent | 2.22 | 1.15 | 2.57E−12 | 3.31E−11 |
| C3ar1 | Complement component 3a receptor 1 | 2.16 | 1.11 | 4.41E−08 | 2.52E−07 |
| Cfp | Complement factor properdin | 2.00 | 1.00 | 2.82E−05 | 6.88E−05 |

[symbol]

indicates data missing or illegible when filed

TABLE 1.4
Regulators of Cytokines, Chemokines and Interleukins

| Gene | | | | | |
|---|---|---|---|---|---|
| symbol | Description | fold Change | log2 Fold C [symbol] | p-value | adj. p-value |
| Socs5 | Suppressor of cytokine signalling 5 | 8.37 | 3.07 | 6.16E−126 | 4.67E−123 |
| Il13ra2 | Interleukin 13 receptor, alpha 2 | 3.90 | 1.97 | 5.14E−07 | 2.13E−06 |
| Irf8 | interferon regulator factor 8 | 3.28 | 1.71 | 1.03E−06 | 3.82E−06 |
| Irf4 | interferon regulatory factor 4 | 3.26 | 1.71 | 5.43E−04 | 1.00E−03 |
| Il21r | interleukin 21 receptor | 3.20 | 1.68 | 2.16E−05 | 5.42E−05 |
| Ccr5 | chemokine (C-C motif) receptor 5 | 3.20 | 1.68 | 3.01E−05 | 7.27E−05 |
| Ifi205 | interferon activated gene 205 | 2.99 | 1.58 | 7.29E−04 | 1.32E−03 |
| Cx3cr1 | chemokine (C-X3-C) receptor 1 | 2.88 | 1.53 | 6.34E−24 | 2.60E−22 |
| Igtp | interferon gamma induced GTPase | 2.83 | 1.50 | 5.09E−05 | 1.17E−04 |
| Socs3 | suppressor of cytokine signalling 3 | 2.49 | 1.32 | 6.73E−05 | 1.50E−04 |
| Il18r1 | interleukin 18 receptor 1 | 2.47 | 1.31 | 2.16E−07 | 1.03E−06 |
| Irak3 | interleukin-1 receptor-associated kinase 3 | 2.43 | 1.28 | 3.80E−11 | 4.03E−10 |
| Cmklr1 | chemokine-like receptor 1 | 2.42 | 1.27 | 6.49E−07 | 2.59E−06 |
| Il1r1 | interleukin 1 receptor, type 1 | 2.36 | 1.24 | 1.87E−19 | 5.46E−18 |
| Ifi203 | interferon activated gene 203 | 2.30 | 1.20 | 4.15E−04 | 7.73E−04 |
| Il1rl1 | Interleukin 1 receptor-like 1 | 2.27 | 1.18 | 2.81E−04 | 5.42E−04 |
| Il18bp | Interleukin 18 binding protein | 2.20 | 1.14 | 3.58E−07 | 1.58E−06 |
| Il10ra | Interleukin 10 receptor, alpha | 2.18 | 1.12 | 2.71E−07 | 1.24E−06 |
| Il13ra1 | Interleukin 13 receptor, alpha 1 | 2.01 | 1.01 | 1.26E−11 | 1.45E−10 |

[symbol]

indicates data missing or illegible when filed

TABLE 1.5
Visual cycle and ion channels

| Gene | | | | | |
|---|---|---|---|---|---|
| symbol | Description | fold Change | log2 Fold C [symbol] | p-value | adj. p-value |
| Cnga1 | cyclic nucleotide gated channel alpha 1 | 16.17 | 4.02 | 4.61E−16 | 8.85E−15 |
| Gnat1 | Guanine nucleotide binding protein, alpha transducing 1 | 10.36 | 3.37 | 8.86E−15 | 1.49E−13 |
| Pde6c | Phosphodiesterase 6C, cGMP-specific, cone, alpha prime | 10.10 | 3.34 | 2.90E−16 | 5.86E−15 |
| Pde6d | Phosphodiesterase 6D, cGMP-specific, rod, delta | 5.84 | 2.55 | 1.51E−10 | 1.40E−09 |
| Clca2 | Chloride channel accessory 2 | 4.32 | 2.11 | 2.22E−03 | 3.76E−03 |
| Rdh1 | Retinol dehydroginase 1 (all trans) | 4.25 | 2.09 | 3.19E−09 | 2.37E−08 |
| Grk1 | G protein-coupled receptor kinase 1 | 3.59 | 1.84 | 6.64E−22 | 2.19E−20 |
| Pde6g | Phosphodiesterase 6G, cGMP-specific, rod, gamma | 3.56 | 1.83 | 2.42E−07 | 1.14E−06 |
| Cnga3 | cyclic nucleotide gated channel alpha 3 | 3.45 | 1.79 | 7.22E−09 | 4.93E−08 |
| Opn4 | Opsin 4 (melanopsin) | 2.92 | 1.55 | 4.34E−12 | 5.49E−11 |
| Rbp7 | retinal binding protein 7, cellular | 2.77 | 1.47 | 2.29E−30 | 1.65E−28 |
| Htr3a | 5-hydroxytryptamine (serotonin) receptor 3A, iontropic | 2.68 | 1.42 | 1.17E−16 | 2.47E−15 |
| Cacng1 | calcium channel, voltage-dependent, gamma subunit 1 | 2.24 | 1.16 | 1.44E−05 | 3.79E−05 |
| Gnb3 | guanine nucleotide binding protein, beta polypeptide 3 | 2.14 | 1.10 | 1.31E−22 | 4.62E−21 |
| Gngt2 | guanine nucleotide binding protein, gamma transducing activity polypeptide 2 | 2.09 | 1.07 | 3.32E−10 | 2.91E−09 |
| Grin3a | glutamate receptor, ionotropic, N-methyl-D-aspartate 3A | 0.49 | −1.03 | 4.73E−06 | 1.43E−05 |
| Chrna6 | cholinergic receptor, nicotinic, alpha 6 (neuronal) | 0.46 | −1.11 | 1.28E−17 | 3.03E−16 |
| Fxyd4 | FXYD domain containing ion transport regulator 4 | 0.43 | −1.21 | 1.11E−02 | 1.71E−02 |
| Trpc7 | Transient receptor potential cation channel, subfamily C, member 7 | 0.40 | −1.32 | 2.72E−02 | 3.79E−02 |
| Gpr50 | G protein-coupled receptor 50 | 0.39 | −1.36 | 4.87E−03 | 7.91E−03 |
| Lrat | lecithin retinol acyltransferase | 0.30 | −1.73 | 1.10E−32 | 1.05E−30 |
| Kcng2 | potassium voltage-gated channel, subfamily G, member 2 | 0.30 | −1.75 | 3.29E−03 | 5.48E−03 |
| Gpr101 | G protein-coupled receptor 101 | 0.22 | −2.17 | 4.77E−03 | 7.78E−03 |

[symbol]

indicates data missing or illegible when filed

All publications mentioned in the above specification are herein incorporated by reference. Various modifications and variations of the described compounds, uses and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. A method of treating or preventing retinitis pigmentosa, wherein the method comprises administering an adeno-associated virus (AAV) vector comprising a nucleotide sequence encoding ciliary neurotrophic factor (CNTF) to a subject in need thereof.

2. The method of claim 1, wherein the AAV vector comprises an AAV serotype 2 genome.

3. The method of claim 1, wherein the CNTF is human CNTF.

4. The method of claim 1, wherein the AAV vector comprises a secretion signal sequence operably linked to the CNTF-encoding nucleotide sequence.

5. The method of claim 4, wherein the secretion signal sequence is a human neuronal growth factor (NGF) secretion signal sequence.

6. The method of claim 1, wherein the AAV vector is administered to the eye of a subject by subretinal, direct retinal or intravitreal injection.

7. The method of claim 1, wherein the AAV vector is administered to the eye of a subject by subretinal injection.

8. The method of claim 1, wherein the AAV vector is administered to a subject in a single dose.

9. The method of claim 1, wherein the subject substantially lacks rod cells in the eye to be treated at the time of administration of the AAV vector.

10. The method of claim 1, wherein photoreceptor cell degeneration due to retinitis pigmentosa is substantially prevented for the lifetime of the subject.

11. The method of claim 10, wherein cone cell degeneration due to retinitis pigmentosa is substantially prevented for the lifetime of the subject.

12. A method of reducing photoreceptor cell death in a subject suffering from or at risk of developing retinitis pigmentosa, wherein the method comprises administering an adeno-associated virus (AAV) vector comprising a nucleotide sequence encoding ciliary neurotrophic factor (CNTF) to a subject in need thereof and wherein visual function is substantially restored or maintained in the treated eye.

13. The method of claim 12, wherein photoreceptor cell degeneration due to retinitis pigmentosa is substantially prevented for the lifetime of the subject.

14. The method of claim 13, wherein cone cell degeneration due to retinitis pigmentosa is substantially prevented for the lifetime of the subject.

15. The method of claim 12, wherein the subject substantially lacks rod cells in the eye to be treated at the time of administration of the AAV vector.


SEQUENCE LISTING (13 sequences)

SEQ ID NO: 1 - DNA, 603 bp, Homo sapiens
atggctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatgtag

SEQ ID NO: 2 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 3 - DNA, 54 bp, Artificial Sequence
  Secretion signal sequence
atgtccatgttgttctacactctgatcacagcttttctgatcggcatacaggcg

SEQ ID NO: 4 - DNA, 934 bp, Artificial Sequence
  Promoter sequence
attgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 5 - DNA, 270 bp, Artificial Sequence
  Bovine Growth Hormone poly-A signal sequence
tcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 6 - DNA, 588 bp, Artificial Sequence
  Woodchuck hepatitis postregulatory element (WPRE) sequence
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 7 - DNA, 66 bp, Artificial Sequence
  Secretion signal sequence
atgtccatgttgttctacactctgatcacagcttttctgatcggcatacaggcggaaccacactca

SEQ ID NO: 8 - DNA, 666 bp, Artificial Sequence
  Human NGF secretion signal sequence operably linked to a human CNTF sequence
atgtccatgttgttctacactctgatcacagcttttctgatcggcatacaggcggaaccacactcagctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatgtag

SEQ ID NO: 9 - protein, 221 aa, Artificial Sequence
  Human NGF secretion signal sequence operably linked to a human CNTF sequence
MSMLFYTLITAFLIGIQAEPHSAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 10 - DNA, 27 bp, Artificial Sequence
  Sense primer
gtacgcggccgcatgtccatgttgttc

SEQ ID NO: 11 - DNA, 26 bp, Artificial Sequence
  Antisense primer
gctctgtgaaagctgagtgtggttcc

SEQ ID NO: 12 - DNA, 26 bp, Artificial Sequence
  Sense primer
ggaaccacactcagctttcacagagc

SEQ ID NO: 13 - DNA, 23 bp, Artificial Sequence
  Antisense primer
tctagtcgacctacattttcttg